(19) United States
(12) Patent Application Publication
(10) Pub. No.: US 2022/0162559 A1
(43) Pub. Date: May 26, 2022

(54) MYOCARDIAL ORGANOIDS AND METHODS OF MAKING AND USES THEREOF

(71) Applicant: CLEMSON UNIVERSITY RESEARCH FOUNDATION, Clemson, SC (US)

(72) Inventors: Ying Mei, Mount Pleasant, SC (US); Dylan Jack Richards, Charleston, SC (US)

(21) Appl. No.: 17/258,820

(22) PCT Filed: Jul. 9, 2019
(86) PCT No.: PCT/US19/40981

§ 371 Date: Jan. 8, 2021

Related U.S. Application Data
(60) Provisional application No. 62/696,660, filed on Jul. 11, 2018.

(51) Int. Cl. C12N 5/077; C12N 5/071

(52) U.S. Cl. CPC C12N 5/0657; C12N 5/0697; C12N 2501/165; C12N 2502/1329; C12N 2506/45; C12N 2513/00

(57) ABSTRACT

This invention relates to three-dimensional myocardial infarct organoids and methods of making and using the same for screening compounds that improve cardiac function and compounds that diminish cardiac function.

### STATEMENT OF PRIORITY

This patent application is a 35 U.S.C. § 371 national phase application of PCT Application PCT/US2019/040981 filed Jul. 9, 2019, which claims the benefit, under 35 U.S.C. § 119(e), of U.S. Provisional Application No. 62/696,660, filed on Jul. 11, 2018, the entire contents of each of which are incorporated by reference herein.

### STATEMENT OF PRIORITY

This invention generally relates to a three-dimensional myocardial infarct organoids and methods of making and using the same.

### FIELD OF THE INVENTION

This invention generally relates to three-dimensional myocardial infarct organoids and methods of making and using the same.

### BACKGROUND OF THE INVENTION

While human organoid systems have provided a powerful platform in modeling diseases caused by genetic disorders¹⁻⁴, non-genetic factors (such as lifestyle and environment) are the largest attributors to devastating diseases like cardiovascular disease (CVD), which is the leading cause of death worldwide.⁵ Specifically, myocardial infarction (MI) (i.e., heart attack) makes up about 8.5% of CVD and is a common cause of heart failure with a 40% five-year mortality after the first MI.⁵ Heart failure drugs have performed poorly in clinical trials during the last decade, which has been partially attributed to the distinct differences between human patient hearts and animal heart failure models.⁷⁻⁹ In addition, cardiotoxicity is a major concern for pre- and post-approval in the development for all systemically-delivered drugs.³² Specifically, the ability to detect drug-induced exacerbation of cardiotoxicity is an unmet need for all drug development to address safety concerns for patients with pre-existing cardiovascular conditions, as CVD is a common comorbidity of major diseases.³²⁻³⁵ Thus, there is a need to develop relevant human heart failure models for drug development.⁶

The present invention overcomes the shortcomings in the field by providing methods of making three-dimensional (3D) myocardial infarct organoids, which can be employed in drug screening and in personalized medicine related to cardiac disease.

### SUMMARY OF THE INVENTION

One aspect of the invention relates to a three-dimensional (3D) myocardial infarct organoid, comprising cardiomyocytes and non-myocytes, wherein the 3D myocardial infarct organoid comprises:

(a) an apoptotic interior region due to lack of oxygen that is surrounded by a viable periphery comprising, consisting essentially of, or consisting of a region of about 20-75 μm from the organoid edge.

(b) a ratio of a terminal deoxynucleotidyl transferase dUTP nick end labeling (TUNEL)-positive area to a 4′,6-diamidino-2-phenylindole (DAPI)-positive area ratio in the apoptotic interior region of in a organoid cross-section ranging from about 0.03 to 1.0;

(c) a contraction amplitude from about 0% to 5%;

(d) a beat rate of about 0 to 90 beats per minute;

(e) a calcium transient amplitude measured as a change in fluorescence divided by the starting fluorescence of about 0% to 40%;

(f) upregulated or downregulated fibrosis-related genes, wherein the downregulated genes comprise COL22A1, COL11A2, FGF12, SPINT1, COL9A2, MMP13, HPN, CTSS, FGF7, A2M, COL9A1, FBN2, FGF9, LAMA3, FLT1, SMOC2, COL2A1, FGF2, LAMB3, LAMA5, PDGFB, SMAD3, ICAM5, LAMB2, FGF18, MMP9, CXCL12, COL19A1, FGF13, COL4A5, COL26A1, F11R, COL14A1, COL9A3, FGF1, ICAM1, HBEGF, MDK, ITGA6, TGFB3, LAMA2, RHOQ, RND2, TGFB2, LAMC2, CCDC88A, ITGAE, JUP, ITGAM, COL4A2, CDH2, ITGB2, TGFBR3, BTG1, COL4A1, COL4A3, PDGFRA, FGFR4, SDC4, MMPI, FGF6, ITGA8, and/or COL4A4,

and upregulated genes comprise FGF8, ITGB1BP1, ITGA7, TGIF2, MMP12, PIK3CA, RHOH, COL18A1, ITGAL, LAMC1, RPS6KB1, TGFBR2, RHOD, PIK3CD, MMP3, RAC1, MMP8, ITGB4, ARPCSL, ITGA3, COL17A1, ADAM9, CD2AP, KDR, LAMB1, COL12A1, ITGAX, ABI1, MMP15, FGF14, TGFB1I1, SDC3, ITGAV, FGFR1, TNC, FGF11, FGFR3, RHOJ, LAMA4, FBLN1, CTSL, DDR2, PDGFRB, MMP24, CD151, ACAN, RHOU, ARF4, COL3A1, FGFR2, COL7A1, ADAM15, CD47, COL10A1, VTN, RHOG, CAPN2, BGN, CXCR4, HTRA1, ICAM2, JAM3, ANG, TGIF1, ITGB7, CD63, RHOA, RHOC, ITGA2, DPP4, COL6A3, COL15A1, SDC2, SPOCK2, DCN, BCAN, COL13A1, ITGB1, MATN3, CLDN1, TIMP2, ARPC5, FN1, CST3, TPM3, MATN1, CD44, HAPLN1, SERPINH1, TIMP3, ITGB3, PLOD3, L1CAM, COL11A1, SPARC, COL6A2, FGF10, P4HA1, IBSP, GREM1, COL6A1, HAS1, CTGF, BMP1, RHOB, VCAN, TGFBR1, MMP10, COL5A2, MFAP2, FGF5, DPT, COL8A1, ITGB5, BDKRB1, COL1A2, TGFB1, MMP11, SERPINE1, LOXL2, FSCN1, SPP1, ITGA4, POSTN, COL5A1, RELN, MMP16, CCDC80, LAMA1, COL1A1, FBN1, ITGA5, LOX, MMP17, LOXL1, LCP1, SDC1, MMP2, MMP14, FAP, TNXB, TGFBI, HAS2, MFAP5, and/or CTSK;

(g) upregulated or downregulated calcium signaling-related genes comprising genes from the Kyoto Encyclopedia of Genes and Genomes (KEGG) calcium signaling pathway, wherein the downregulated genes comprise CACNA1G, EDNRB, CHRM1, ADRB1, PLCG2, ERBB4, RYR3, ERBB3, ATP2A2, ADRB2, P2RX7, PLCB1, ATP2A1, CAMK2A, RYR2, HRH2, PHKA1, PHKG1, ATP2B2, PDE1C, HTR4, CACNA1C, CAMK2B, SLC8A1, SLC25A5, CACNA1S, P2RX1, TBXA2R, CAMK2D, PRKACA, PHKA2, GRIN2C, PPIF, ADCY9, PTK2B, VDAC3, EGFR, VDAC2, PHKB, NOS2, PLCD1, GRIN2A, CALML4, P2RX6, TNNC2, VDAC1, PHKG2, CHRNA7, PRKCB, GRPR, SLC25A4, NOS1, CCKBR, ADORA2A, ADCY3, NTSR1, GRIN1, ADRA1A, PDGFRA, PPP3CB, NOS3, HTR2C, MYLK, TNNC1, and/or PLCG,

and the upregulated genes comprise GNA15, CACNA1H, GNAS, HTR5A, PTGFR, PTGER1, TACR1, RYR1, PRKACB, CCKAR, CD38, PTAFR, CALM2, PDE1A, PPP3R1, LHCGR, ADCY2, TACR2, PLCB3, GNA11, BDKRB2, PRKCG, STIM1, ADCY4, ATP2A3, GNA14, AVPR1A, CACNA1B, ITPR2, PPP3CC, HTR7, HTR2B, PPP3CA, PDGFRB, SPHK2, PRKCA, GRIN2D, PDE1B, GNAQ, CALM1, ITPKB, HRH1, CAMK4, P2RX4, PTGER3, ITPR1, ADCY7, ADORA2B, F2R, CACNA1E, BDKRB1, SPHK1, CACNA1A, ADRA1B, ADRB3, ITPR3, and/or ADCY8; and/or

(h) an elastic modulus of about 3 kPa to about 5 kPa.

A second aspect provides a method of making a 3D myocardial infarct organoid, the method comprising: culturing cardiomyocytes with non-myocytes for about 1 day to 20 days to form a self-assembled 3D cardiac organoid under normoxic conditions; and exposing the 3D cardiac organoid to hypoxic conditions for about 1 day to 20 days, thereby generating the 3D myocardial infarct organoid.

A third aspect of the invention provides a method of making a 3D myocardial ischemia-reperfused organoid, the method comprising: culturing cardiomyocytes with non-myocytes for about 1 day to 20 days to form a 3D cardiac organoid under normoxic conditions; exposing the 3D cardiac organoid under hypoxic conditions for about 1 day to 20 days to form a 3D myocardial infarct organoid, and exposing the 3D myocardial infarct organoid to normoxic conditions (and/or fresh culture media) for about 5 seconds to 20 days, thereby generating the 3D myocardial ischemia-reperfused organoid.

Further provided are methods for screening a compound for improving or diminishing cardiac function.

These and other aspects of the invention are set forth in more detail in the description of the invention below.

### DESCRIPTION OF THE DRAWINGS

FIGS. 1A-1F show cardiac infarct organoids model human myocardial infarction using major upstream pathological stimuli. FIG. 1A shows the 3D nature and diffusion limitations in the post-myocardial infarction (MI) heart can be spatially mimicked in cardiac organoid culture to create an in vitro myocardial infarction model. FIGS. 1B and 1C show finite element modeling and quantification of oxygen diffusion in simulated cardiac microtissues revealing the inherent diffusion limitation of oxygen in microtissues at 20% and 10% external oxygen. FIG. 1D shows NADH autofluorescence from live two-photon imaging (>30 μm below surface) of live control, infarct, and dead (frozen+thaw) cardiac organoids and NADH index quantification showing lower NADH in the center of organoids and overall lower levels in infarct organoids. *p<0.05 using one-way ANOVA with Bonferroni-corrected t-test post-hoc. n=5 organoids from 1 experiment. FIG. 1E, shows overlap of differentially expressed (DE) (p<0.05) genes from infarct organoids (vs. control organoids) RNA sequencing data compared to human ischemic cardiomyopathy (vs. nonfailing), mouse 1 week post-MI (vs. sham), and pig 1 week post-MI (vs. sham) microarray data. FIG. 1F shows principal component analysis of the 4,765 shared genes between the cardiac organoid samples and mouse 2 week post-MI and human ischemic cardiomyopathy RNA sequencing samples.

FIGS. 2A-2J show characterization of fibrosis in cardiac infarct organoids at the transcriptomic, structural, and functional level. FIGS. 2A-2C show gene ontology terms (FIG. 2A) and fibrosis-related gene sets (FIGS. 2B and 2C) in organoid model showing similar trends in gene expression changes after injury compared to mouse 1 week post-myocardial infarction (MI) microarray data. Organoid samples, n=3 biological replicates (30-35 organoids per replicate); mouse, n=3 biological replicates. FIG. 2D shows vimentin radial density. n=15 sections of separate organoids per group across 3 individual experiments. Mean±standard error mean. Student's t-test was used for statistical significance. FIG. 2E shows vimentin radial density plots of representative vimentin immunofluorescent staining of infarct organoid sections with or without “anti-fibrotic” (JQ1, 10 nM) culture conditions. n=10,10,7 (control, infarct, JQ1) sections of separate organoids across 2 individual experiments. Mean±standard error mean. Student's t-test was used for statistical significance.

FIG. 2F shows stiffness (i.e., elastic modulus, kPa) calculated using equilibrium deformation displacement. n=19, 21 (control, infarct) organoids from 3 individual experiments. *p<0.05 using Student's t-test. FIG. 2G shows percent change in elastic modulus relative to control for cardiac infarction protocol with added “pro-” (TGF-β1, 20 ng/ml) or “anti-fibrotic” (JQ1, 10 nM) culture conditions. n=7,6,5 (infarct, pro-fibrotic, anti-fibrotic) organoids from 1 experiment. *p<0.05 using one-way ANOVA with Bonferroni-corrected t-test post-hoc. FIG. 2H shows a heatmap of DE genes in the “metabolic pathway” (KEGG Pathway map01100) in organoid model showing similar trends in gene expression changes after injury compared to mouse 1 week post-MI microarray data. Scale is row z-score. Organoid samples, n-3 biological replications (30-35 organoids per replicate); mouse, n=3 biological replicates. FIG. 2I shows top identified pathways from organoid RNA sequence data. FIG. 2J shows representative fibrosis-related genes from organoid RNA sequencing indicating significant changes in infarct organoids. *p<0.05 using DESeq2 differential expression analysis of sequencing data.

FIGS. 3A-3F show characterization of pathological calcium-handling in cardiac infarct organoids at the transcriptomic, structural, and functional level. FIGS. 3A-3B show calcium handling-related gene set in organoid model showing similar trends in gene expression changes after injury compared to mouse 1 week post-myocardial infarction (MI) microarray data. Organoid samples, n=3 biological replicates (30-35 organoids per replicate); mouse, n=3 biological replicates. FIG. 3C shows quantification of calcium transient amplitude (ΔF/F₀) of separate ROIs representing individual cardiomyocytes from selected imaging planes at >50 μm below organoid surface. n=32 ROIs across 10 control organoids, 47 edge ROIs and 35 interior ROIs across 19 infarct organoids all across 3 individual experiments. Mean±standard error mean. *p<0.05 using one-way ANOVA with Bonferroni-corrected t-test post-hoc. FIG. 3D shows the proportion of organoids (control, infarct, infarct with anti-fibrotic treatment (JQ, 10 nM), and infarct with pro-angiogenic treatment (human recombinant vascular endothelial growth factor-VEGF, 2 ng/ml) that exhibited synchronized or unsynchronized beating, n=34-35 organoids per group. FIG. 3E shows percent change in elastic modulus relative to control on D10 for cardiac infarction protocol with added “anti-fibrotic” (JQ1, 10 nM) culture conditions. n=13-14 organoids per group from 2 individual experiments. FIG. 3F shows representative calcium-related genes from organoid RNA sequencing indicating significant change in major calcium handling genes. *p<0.05 using DESeq2 differential expression analysis of sequencing data.

FIGS. 4A-4D show development of cardiac organoid infarction protocol. FIG. 4A shows beat rate of cardiac organoids on D10. n=37, 39, 15 across 6, 6, 2 individual experiments for control, infarct, and infarct+metoprolol (10 μM) organoids, respectively. Mean±standard error mean. *p<0.05 using one-way ANOVA with Bonferroni-corrected t-test post-hoc. FIG. 4B shows contraction amplitude (fractional area change) on D10. n=30 organoids per group across 5 individual experiments. Mean±standard error mean. *p<0.05 using Student's t-test. FIG. 4C shows diameters of organoids on D0 and D10. n=152-252 organoids per group from 3 individual experiments. FIG. 4D shows NADH index quantification (mean±standard deviation) of live control, infarct, and dead (frozen and thawed) cardiac organoids on D10 showing lower NADH in the center of organoids and overall lower levels in infarct organoids. *p<0.05 using one-way ANOVA with Bonferroni-corrected t-test post-hoc. n=10-11 organoids per group from 2 individual experiments.

FIGS. 5A-5C show meta-analysis using principal component analysis (PCA) of cardiac injury studies. FIG. 5A shows a boxplot of individual samples in principal components 1 (PC1) to PC10 from RNA sequencing or cardiac organoid sand human ischemic cardiomyopathy and mouse MI studies. FIG. 5B shows cumulative proportion of variance for all 30 PCs with a zoom-in on PC1-10. FIG. 5C shows a boxplot of individual samples of PC1 and PC2 with the addition of separate mouse sham heart RNA-seq data (ms96561) revealing distinction between species and tissue complexity (i.e., organoid- vs organ-derived) (left) and lack of variation due to sequencing platform (right).

FIG. 6 shows elastic modulus of microtissue variants. Spheroids/organoids formed using human induced pluripotent stem cell-derived cardiomyocytes (CM) only, cardiac fibroblasts (FB) only, cardiac organoids, or cardiac organoids with an additional 10% FB (organoid+FB) and measured on Day 0 using micropipette aspiration to appreciate cell composition contributions to changes in stiffness. n=6, 5, 6, 4 microtissues (CM spheroid, FB spheroid, cardiac organoid, organoid+FB). *p<0.05 using one-way ANOVA with Bonferroni-corrected t-test post-hoc.

FIG. 7 shows calcium transient quantification of human induced pluripotent stem cell-derived cardiomyocyte (hiPSC-CM) spheroids imaged in situ with customized Two-Photon scanned Light Sheet Microscope (2PLSM). Quantification of calcium transient amplitude (ΔF/F₀) of separate ROIs representing individual cardiomyocytes from selected imaging planes at >50 μm below surface of live GCaMP6-labeled hiPSC-CM spheroids on Day 10 labeled with fluorescent indicator in control or organoid infarction culture conditions. n=8 ROIs across 3 control spheroids, 12 edge ROIs and 11 interior ROIs across 4 infarct spheroids. Mean±standard error mean.

FIGS. 8A-8D show detection of tissue-level drug-induced exacerbation of cardiotoxicity using cardia infarct organoids. FIG. 8A shows normalized contraction amplitude relative to vehicle control of each group, with IC₅₀ or organoids in response to a range of doxorubicin (DOX) (0-50) after 48 hrs of exposure starting on D10. n=7-14 organoids per dose from 2 individual experiments. For box-plots, center line—median; box limits—upper and lower quartiles; whiskers—total range. FIG. 8B shows normalized viability index relative to vehicle control of each group, based on TUNEL-apoptotic staining or organoid sections at a range of DOX doses (0-10 μM) after 48 hours of exposure starting on D10. n=6-10 organoids per group from 2 individual experiments. *p<0.05 using one-way ANOVA with Bonferroni-corrected t-test post-hoc. FIG. 8C shows changes in sarcomeric organization caused by increased dose of DOX (48 hrs of exposure starting on D10) quantified by radial density of alpha sarcomeric immunofluorescent staining in organoid sections. n=5-8 organoids per dose from 2 individual experiments. -p <0.05 for 0.1 μM versus 0 μM DOX; x p<0.05 for 1.0 μM versus 0 μM DOX using Student's t-test. FIG. 8D shows DOX-induced changes in vimentin-covered area relative to vehicle control of each group, in organoid sections after 48 hrs of exposure starting on D10. n=7-10 organoids per group from 2 individual experiments. *p<0.05 using one-way ANOVA with Bonferroni-corrected t-test post-hoc.

### DETAILED DESCRIPTION OF THE INVENTION

The present invention now will be described hereinafter with reference to the accompanying drawings and examples, in which embodiments of the invention are shown. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein, when referring to a measurable value such as an amount or concentration and the like, is meant to encompass variations of ±10%, ±5%, ±1%, ±0.5%, or even ±0.1% of the specified value as well as the specified value. For example, “about X” where X is the measurable value, is meant to include X as well as variations of ±10%, ±5%, ±1%, ±0.5%, or even ±0.1% of X. A range provided herein for a measureable value may include any other range and/or individual value therein.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

As used herein, the terms “increase,” “increasing,” “increased,” “enhance,” “enhanced,” “enhancing,” and “enhancement” (and grammatical variations thereof) describe an elevation of at least about 25%, 50%, 75%, 100%, 150%, 200%, 300%, 400%, 500% or more as compared to a control.

As used herein, the terms “reduce,” “reduced,” “reducing,” “reduction,” “diminish,” and “decrease” (and grammatical variations thereof), describe, for example, a decrease of at least about 5%, 10%, 15%, 20%, 25%, 35%, 50%, 75%, 80%, 85%, 90%, 95%, 97%, 98%, 99%, or 100% as compared to a control. In particular embodiments, the reduction can result in no or essentially no (i.e., an insignificant amount, e.g., less than about 10% or even 5%) detectable activity or amount.

As used herein, the term “cardiomyocytes” refers to cardiac muscle cells that make up the cardiac muscle (heart muscle). Each myocardial cell contains myofibrils, which are specialized organelles consisting of long chains of sarcomeres, the fundamental contractile units of muscle cells. Cardiomyocytes show striations similar to those on skeletal muscle cells. Unlike multinucleated skeletal cells, the majority of cardiomyocytes contain only one nucleus, although they may have as many as four. Cardiomyocytes have a high mitochondrial density, which allows them to produce adenosine triphosphate (ATP) quickly, making them highly resistant to fatigue.

As used herein, the term “non-myocytes” refer to cells that are generally responsible for transmitting biochemical, mechanical and electrical cues, which makes them essential components in the cardiac microenvironment. Examples include, but are not limited to, fibroblasts (FBs), stem cells (e.g., human adipose derived stem cells (hADSCs)), endothelial cells (ECs) (e.g., human umbilical vein endothelial cells (HUVECs)), smooth muscle cells, neurons and immune cells, or any combination thereof.

As used herein, the term “contraction amplitude” refers to the ability of the cardiomyocytes present in a myocardial organoid and/or myocardial infarct organoid and/or myocardial ischemic-reperfused organoid to contract. Generally, the contraction amplitude of a heart measures the ability of a cardiac muscle to contract, which is essential for pushing blood through the heart and/or body of a mammal and is therefore a relevant measurement for the cardiac organoid. Specifically, for spherical microtissues, like cardiac organoids, contraction amplitude is measured from the percent change in fractional projected area change from peak contraction to relaxation calculated from videos of contraction.

As used herein, the term “beat rate” refers to the number of contractions per minute (bpm) of the cardiomyocytes in an organoid.

As used herein, the term “calcium transient amplitude” refers to the changes in calcium fluorescence signal as measured by fluorescent calcium probes, including but not limited to GCaMP6, indicating the relative calcium concentration in the organoid as the cardiomyocytes in the organoid contract and/or relax. In general, Ca²⁺ is released from the sarcoplasmic reticulum (SR) resulting in the efflux of Ca²⁺ from the SR into the cytoplasm resulting in contraction of the cardiomyocytes in the organoid. Relaxation is initiated by a reduction of [Ca²⁺] produced either by pumping back into the SR by the SR Ca²⁺-ATPase (SERCA) or out of the cell, largely by the sarcolemmal NatCa²⁺ exchange.

As used herein, the term “elastic modulus” describes the degree of stiffness and/or elasticity of a tissue. For myocardial tissue an increase in elastic modulus, i.e., stiffness, prevents contraction of the cardiomyocytes in the organoid and thus results in a decrease in cardiac function.

As used herein, the term “DAPI” stands for 4′,6-diamidino-2-phenylindole, which is a fluorescent stain that binds strongly to adenine-thymine rich regions in DNA. It is used extensively in fluorescence microscopy. As DAPI can pass through an intact cell membrane, it can be used to stain both live and fixed cells, though it passes through the membrane less efficiently in live cells and therefore the effectiveness of the stain is lower. Thus a “DAPI-positive area” would be the total area that stains positive for DAPI per organoid after fixation and permeabilization on 7 μm thickness frozen cross sections of cardiac organoids.

As used herein, the term “TUNEL” stands for terminal deoxynucleotidyl transferase dUTP nick end labeling, which is a method for detecting DNA fragmentation by labeling the 3′- hydroxyl termini in the double-strand DNA breaks generated during apoptosis. Thus, the TUNEL method may be used to detect apoptotic DNA fragmentation, therefore, may be used to identify and quantify apoptotic cells, or to detect excessive DNA breakage in individual cells. The assay relies on the use of terminal deoxynucleotidyl transferase (TdT), an enzyme that catalyzes attachment of deoxynucleotides, tagged with a fluorochrome or another marker, to 3′-hydroxyl termini of DNA double strand breaks. It may also be used to label cells in which the DNA is damaged by other means than in the course of apoptosis. Thus a TUNEL-positive area” would be an area that stains positive for TUNEL per organoid after fixation and permeabilization on 7 μm thickness frozen cross sections of cardiac organoids.

In the disclosure, the inventors combined non-genetic causal factors of MI with their previously established cardiac organoids to create the first human organoid model of cardiac infarction.^{10,11} In particular, the inventors leveraged the diffusion limitation in 3D microtissues to recreate the nutrient (e.g., oxygen) diffusion gradient across infarcted hearts (i.e., infarct-border-remote zones) in human cardiac organoids to induce cardiac organotypic response to infarction. This enabled the recapitulation of major MI hallmarks in human cardiac organoids at the transcriptomic, structural and functional level.

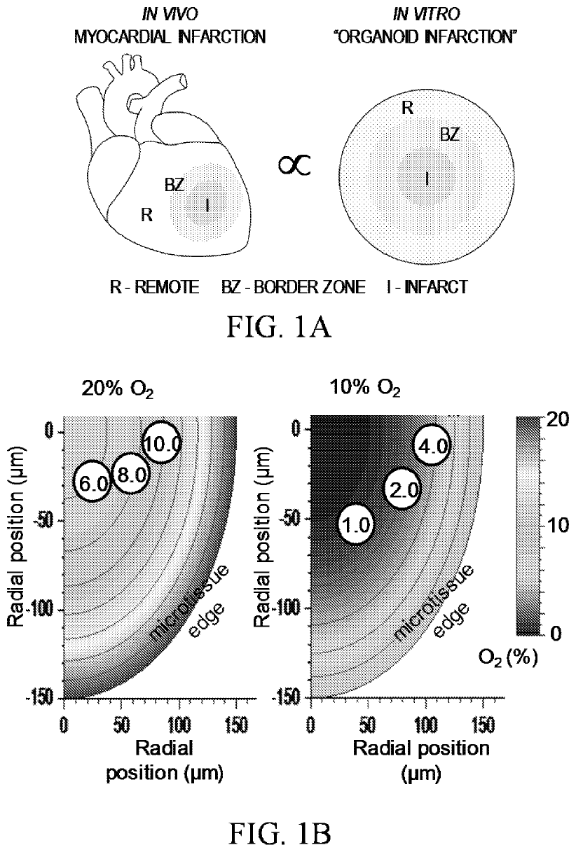

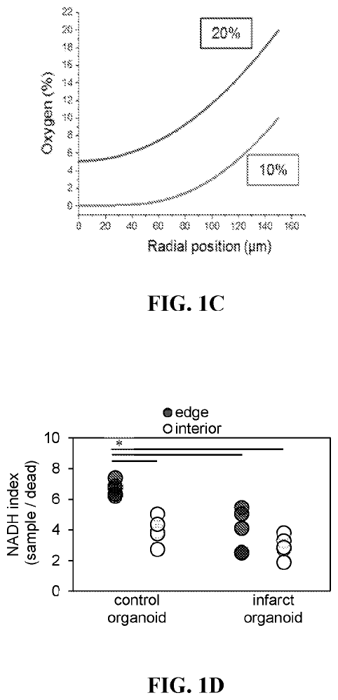

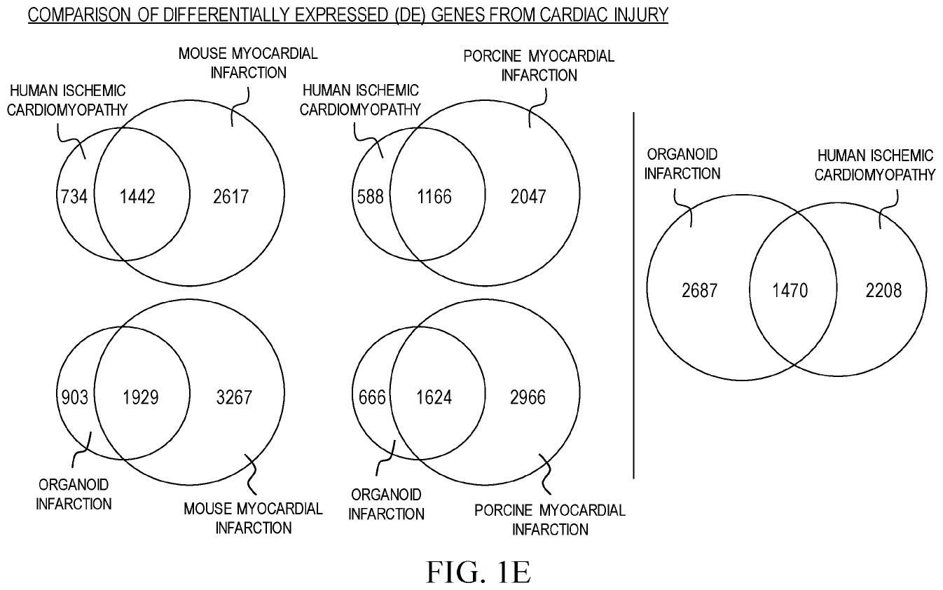

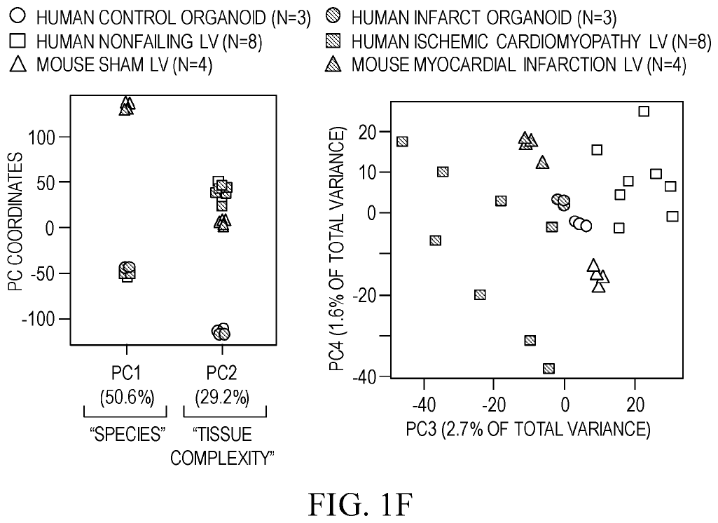

During a heart attack, a blocked artery limits the delivery of blood to downstream myocardium causing massive cell death, leading to reduced ability to pump blood to the body that triggers compensatory efforts by the nervous system to restore cardiac output (i.e., adrenergic stimulation via norepinephrine).¹² Given the inability of the damaged heart to fully compensate or regenerate, this positive feedback causes chronic heart dysfunction and ultimately heart failure.¹² With the understanding of major upstream causal factors in heart failure, the inventors leveraged inherent oxygen diffusion limitations in 3D microtissues and chronic adrenergic stimulation to induce organotypic response of myocardium to infarction with human cardiac organoids (FIG. 1A).

As human cardiac tissues post-MI are difficult to obtain²⁸, human cardiac infarct organoids offer a model of the acute post-infarct heart tissue, a stage that is critical for the understanding the short-term post-MI injured state of both ischemia and ischemia/reperfusion (I/R) caused cardiac injury. While organoids have traditionally been prepared with embryonic bodies, the current disclosure demonstrates that the self-assembly of tissue-specific cell types provides a powerful alternative to prepare organoids with tissue-mimetic transcriptome, structure and function.²⁹

Thus, one aspect of the invention relates to a three-dimensional (3D) myocardial infarct organoid, comprising cardiomyocytes and non-myocytes, wherein the 3D myocardial infarct organoid comprises, consists essentially of, or consists of:

(a) an apoptotic interior region due to lack of oxygen surrounded by a viable periphery that comprises, consists essentially of, or consists of a region of about 20-75 μm from the organoid edge

(b) a ratio of a TUNEL-positive area to a DAPI-positive area ratio in the apoptotic interior region of in a organoid cross-section ranging from about 0.03 to 1.0;

(c) a contraction amplitude from about 0% to 5%;

(d) a beat rate of about 0 to 90 beats per minute;

(e) a calcium transient amplitude measured as a change in fluorescence divided by the starting fluorescence of about 0% to 40%;

(f) upregulated or downregulated fibrosis-related genes', wherein the downregulated genes include, but are not limited to, COL22A1, COL11A2, FGF12, SPINT1, COL9A2, MMP13, HPN, CTSS, FGF7, A2M, COL9A1, FBN2, FGF9, LAMA3, FLT1, SMOC2, COL2A1, FGF2, LAMB3, LAMAS, PDGFB, SMAD3, ICAM5, LAMB2, FGF18, MMP9, CXCL12, COL19A1, FGF13, COL4A5, COL26A1, F11R, COL14A1, COL9A3, FGF1, ICAM1, HBEGF, MDK, ITGA6, TGFB3, LAMA2, RHOQ, RND2, TGFB2, LAMC2, CCDC88A, ITGAE, JUP, ITGAM, COL4A2, CDH2, ITGB2, TGFBR3, BTG1, COL4A1, COL4A3, PDGFRA, FGFR4, SDC4, MMPI, FGF6, ITGA8, and/or COL4A4,

and upregulated genes include, but are not limited to, FGF8, ITGB1BP1, ITGA7, TGIF2, MMP12, PIK3CA, RHOH, COL18A1, ITGAL, LAMC1, RPS6KB1, TGFBR2, RHOD, PIK3CD, MMP3, RAC1, MMP8, ITGB4, ARPCSL, ITGA3, COL17A1, ADAM9, CD2AP, KDR, LAMB1, COL12A1, ITGAX, ABU, MMP15, FGF14, TGFB1I1, SDC3, ITGAV, FGFR1, TNC, FGF11, FGFR3, RHOJ, LAMA4, FBLN1, CTSL, DDR2, PDGFRB, MMP24, CD151, ACAN, RHOU, ARF4, COL3A1, FGFR2, COL7A1, ADAM15, CD47, COL10A1, VTN, RHOG, CAPN2, BGN, CXCR4, HTRA1, ICAM2, JAM3, ANG, TGIF1, ITGB7, CD63, RHOA, RHOC, ITGA2, DPP4, COL6A3, COL15A1, SDC2, SPOCK2, DCN, BCAN, COL13A1, ITGB1, MATN3, CLDN1, TIMP2, ARPCS, FN1, CST3, TPM3, MATN1, CD44, HAPLN1, SERPINH1, TIMP3, ITGB3, PLOD3, L1CAM, COL11A1, SPARC, COL6A2, FGF10, P4HA1, IBSP, GREM1, COL6A1, HAS1, CTGF, BMP1, RHOB, VCAN, TGFBR1, MMP10, COL5A2, MFAP2, FGFS, DPT, COL8A1, ITGB5, BDKRB1, COL1A2, TGFB1, MMP11, SERPINE1, LOXL2, FSCN1, SPP1, ITGA4, POSTN, COL5A1, RELN, MMP16, CCDC80, LAMA1, COL1A1, FBN1, ITGA5, LOX, MMP17, LOXL1, LCP1, SDC1, MMP2, MMP14, FAP, TNXB, TGFBI, HAS2, MFAP5, and/or CTSK;

(g) upregulated or downregulated calcium signaling-related genes comprising genes from the Kyoto Encyclopedia of Genes and Genomes (KEGG) calcium signaling pathway, wherein the downregulated genes include, but are not limited to, CACNA1G, EDNRB, CHRM1, ADRB1, PLCG2, ERBB4, RYR3, ERBB3, ATP2A2, ADRB2, P2RX7, PLCB1, ATP2A1, CAMK2A, RYR2, HRH2, PHKA1, PHKG1, ATP2B2, PDE1C, HTR4, CACNA1C, CAMK2B, SLC8A1, SLC25A5, CACNA1S, P2RX1, TBXA2R, CAMK2D, PRKACA, PHKA2, GRIN2C, PPIF, ADCY9, PTK2B, VDAC3, EGFR, VDAC2, PHKB, NOS2, PLCD1, GRIN2A, CALML4, P2RX6, TNNC2, VDAC1, PHKG2, CHRNA7, PRKCB, GRPR, SLC25A4, NOS1, CCKBR, ADORA2A, ADCY3, NTSR1, GRIN1, ADRA1A, PDGFRA, PPP3CB, NOS3, HTR2C, MYLK, TNNC1, and/or PLCG,

and the upregulated genes include, but are not limited to, GNA15, CACNA1H, GNAS, HTR5A, PTGFR, PTGER1, TACR1, RYR1, PRKACB, CCKAR, CD38, PTAFR, CALM2, PDE1A, PPP3R1, LHCGR, ADCY2, TACR2, PLCB3, GNA11, BDKRB2, PRKCG, STIM1, ADCY4, ATP2A3, GNA14, AVPR1A, CACNA1B, ITPR2, PPP3CC, HTR7, HTR2B, PPP3CA, PDGFRB, SPHK2, PRKCA, GRIN2D, PDE1B, GNAQ, CALM1, ITPKB, HRH1, CAMK4, P2RX4, PTGER3, ITPR1, ADCY7, ADORA2B, F2R, CACNA1E, BDKRB1, SPHK1, CACNA1A, ADRA1B, ADRB3, ITPR3, and/or ADCY8; and/or

(h) an elastic modulus of about 3 kPa to about 5 kPa.

In some embodiments, the cardiomyocytes may comprise induced pluripotent stem cell-derived cardiomyocytes (iPSC-CMs), cardiac progenitor cells, primary cardiomyocytes, or any combination thereof. In some embodiments, the cardiomyocytes and non-myocytes may be present in a ratio of about 95:5 to about 5:95 of cardiomyocytes to non-myocytes. In some embodiments, the cardiomyocytes and non-myocytes may be present in a ratio of about 60:40 to about 40:60 of cardiomyocytes to non-myocytes.

In some embodiments, the non-myocytes may comprise a combination of fibroblasts (FBs), endothelial cells (ECs) and mesenchymal stem cells (MSCs). In some embodiments, the non-myocytes may comprise FBs in amount of about 50% to 60% based on the total number of non-myocytes, ECs in an amount of about 25% to about 35% based on the total number of non-myocytes, and MSCs in an amount of about 10% to about 20% based on the total number of non-myocytes. In some embodiments, the cardiomyocytes and/or non-myocytes are derived from a human.

Another aspect of the invention relates to a method of making a 3D myocardial infarct organoid, the method comprising:

culturing cardiomyocytes with non-myocytes for about 1 day to 20 days to form a self-assembled 3D cardiac organoid under normoxic conditions; and

exposing the 3D cardiac organoid to hypoxic conditions for about 1 day to 20 days, thereby generating the 3D myocardial infarct organoid.

Another aspect of the invention relates to a method of making a 3D myocardial ischemia-reperfused organoid, the method comprising:

culturing cardiomyocytes with non-myocytes for about 1 day to 20 days to form a 3D cardiac organoid under normoxic conditions;

exposing the 3D cardiac organoid under hypoxic conditions for about 1 day to 20 days to form a 3D myocardial infarct organoid; and

exposing the 3D myocardial infarct organoid to normoxic conditions and/or exposing the 3D myocardial infarct organoid to fresh culture media for about 5 seconds to 20 days,

thereby generating the 3D myocardial ischemia-reperfused organoid.

In some embodiments, the cardiomyocytes may be cultured with the non-myocytes at a ratio of about 95:5 to about 5:95 of cardiomyocytes to non-myocytes. In some embodiments, the cardiomyocytes may be cultured with the non-myocytes at a ratio is about 60:40 to about 40:60 of cardiomyocytes to non-myocytes.

In some embodiments, the non-myocytes may comprise fibroblasts (FBs), endothelial cells (ECs), mesenchymal stem cells (MSCs), or any combination thereof. In some embodiments, the non-myocytes may comprise FBs in amount of about 50% to 60% based on the total number of non-myocytes, ECs in an amount of about 25% to about 35% based on the total number of non-myocytes, and MSCs in an amount of about 10% to about 20% based on the total number of non-myocytes. In some embodiments, the ECs may comprise human umbilical vein endothelial cells (HUVECs) and/or MSCs may comprise human adipose derived stem cells (hADSCs).

In some embodiments, the cardiomyocytes and the non-myocytes may be cultured at a total concentration of about 1×10⁵ cells/mL to about 1×10⁷ cells/mL. In some embodiments, the cardiomyocytes and/or non-myocytes are from a human.

In some embodiments, the cardiomyocytes and non-myocytes may be cultured in the presence of norepinephrine, angiotensin II, TNF-alpha, interfering RNAs, microRNAs, matrix metalloproteases, or any combination thereof. In some embodiments, the cardiomyocytes and non-myocytes may be cultured in the presence of norepinephrine at a concentration of about 0.01 μM to about 10 μM.

In some embodiments, the hypoxic conditions may comprise a partial pressure of oxygen in the gas phase that is less than about 15% of the total barometric pressure. In some embodiments, the normoxic conditions may comprise a partial pressure of oxygen in the gas phase of about 16% to about 20.9% of the total barometric pressure.

In some embodiments, the 3D myocardial infarct organoid and/or the 3D myocardial ischemia-reperfused organoid may comprise an average diameter of about 100 μm to about 1000 μm.

Another aspect of the invention relates to a method for screening a compound for improving cardiac function, the method comprising:

contacting the 3D myocardial infarct organoid of the invention or the 3D myocardial ischemia-reperfused organoid of the invention with the compound;

measuring in the 3D myocardial infarct organoid or 3D myocardial ischemia-reperfused organoid the size of an interior apoptotic region, a ratio of a TUNEL-positive area to a DAPI-positive area in the apoptotic region, a contraction amplitude, a beat rate, a calcium transient amplitude, and/or an elastic modulus; and

determining that the compound improves cardiac function when

(a) the interior apoptotic region is reduced by at least about 30% when compared a control;

(b) the ratio of TUNEL-positive area to DAPI-positive area is reduced by at least about 30% when compared to a control;

(c) the contraction amplitude is increased by at least about 30% when compared to a control;

(d) the calcium transient amplitude is increased by about 30% when compared to a control; and/or

(e) the elastic modulus is decreased by about 30% when compared to a control; wherein the control is the 3D myocardial infarct organoid of the invention or the 3D myocardial ischemia-reperfused organoid of the invention that has not been contacted with the compound.

Another aspect of the invention relates to a method for screening a compound for diminishing cardiac function, the method comprising:

contacting the 3D myocardial infarct organoid of the invention or the 3D myocardial ischemia-reperfused organoid of the invention with the compound;

measuring in the 3D myocardial infarct organoid or 3D myocardial ischemia-reperfused organoid the size of an interior apoptotic region, a ratio of a TUNEL-positive area to a DAPI-positive area in the apoptotic region, a contraction amplitude, a beat rate, a calcium transient amplitude, and/or an elastic modulus; and

determining that the compound diminishes cardiac function when

(a) the interior apoptotic region is increased by at least about 30% when compared to a control;

(b) the ratio of TUNEL-positive area to DAPI-positive area is increased by at least 30% when compared to a control;

(c) the contraction amplitude is decreased by at least about 30% when compared to a control;

(d) the calcium transient amplitude is decreased by about 30% when compared to a control; and/or

(e) the elastic modulus is increased by about 30% when compared to a control; wherein the control is the 3D myocardial infarct organoid of the invention or the 3D myocardial ischemia-reperfused organoid of the invention that that has not been contacted with the compound.

In some embodiments, the compound may be a therapeutic compound for treating, for example, cardiovascular disease, diabetes, liver disease, kidney disease, and/or cancer

### I. Three-Dimensional (3D) Myocardial Infarct Organoid Composition

One aspect of the invention relates to a three-dimensional (3D) myocardial infarct organoid comprising cardiomyocytes and non-myocytes, wherein the 3D myocardial infarct organoid can be characterized by one or more of the following characteristics: (a) size of the apoptotic region, (b) ratio of TUNEL-positive area to DAPI-positive area in the apoptotic region, (c) contraction amplitude, (d) beat rate, (e) calcium transient amplitude, (e) upregulated and/or downregulated fibrosis-related genes, (f) upregulated and/or downregulated KEGG calcium signaling pathway genes, and/or (h) elastic modulus.

In some embodiments, the cardiomyocytes include, but are not limited to, induced pluripotent stem cell-derived cardiomyocytes (iPSC-CMs), cardiac progenitor cells, primary cardiomyocytes, or any combination thereof. In some embodiments, non-myocytes include, but are not be limited to, fibroblasts (FBs), endothelial cells (ECs), and mesenchymal cells (MSCs). Exemplary endothelial cells include, but are not limited to, human umbilical vein endothelial cells (HUVECs). Exemplary mesenchymal cells (MSCs) include, but are not limited to, human adipose derived stem cells (hADSCs).

In some embodiments, the cardiomyocytes and/or non-myocytes are from a mammal. A mammal may include but is not limited to a human, a nonhuman primate, a domesticated mammal (e.g., a dog, a cat, a rabbit, a guinea pig, a rat), or a livestock and/or agricultural mammal (e.g., a horse, a bovine, a pig, a goat). In some embodiments, the mammal is a human.

In some embodiments, the cardiomyocytes and non-myocytes are present in a ratio of about 95:5 to about 5:95, about 90:10 to about 10:90, about 85:15 to about 15:85, about 70:30 to about 30:70, or about 60:40 to about 40:60 of cardiomyocytes to non-myocytes (e.g., about 98:2, about 95:5, about 90:10, about 85:15, about 80:20, about 75:25, about 70:30, about 65:35, about 60:40, about 55:45, about 50:50, about 45:55, about 40:60, about 35:65, about 30:70, about 25:75, about 20:80, about 15:85, about 10:90, about 5:95, or about 2:98 of cardiomyocytes to non-myocyte cells.

The amount of fibroblasts (FBs), endothelial cells (ECs), and mesenchymal cells (MSCs) that make up the total amount of non-myocytes present in the 3D myocardial infarct organoid can vary. For example, in some embodiments, the non-myocytes may comprise FBs in amount of about 1% to about 100%, about 20% to about 80%, about 40% to about 70%, or about 50% to about 60% based on the total number of non-myocytes (e.g., about 1%, 2.5%, 5%, 10%, 15%, 20%, 25%, 30%, 35%, 40%, 45%, 50%, 55%, 60%, 65%, 70%, 75%, 80%, 85%, 90%, 95%, 100%, or any value or range therein). In some embodiments, the non-myocytes may comprise ECs in an amount of about 1% to about 100%, about 10% to about 80%, about 20% to about 50%, or about 25% to about 35% based on the total number of non-myocytes (e.g., about 1%, 2.5%, 5%, 10%, 15%, 20%, 25%, 30%, 35%, 40%, 45%, 50%, 55%, 60%, 65%, 70%, 75%, 80%, 85%, 90%, 95%, 100%, or any value or range therein). In some embodiments, the non-myocytes may comprise MSCs in an amount of about 1% to about 100%, about 5% to about 50%, or about 10% to about 20% based on the total number of non-myocytes (e.g., about 1%, 2.5%, 5%, 10%, 15%, 20%, 25%, 30%, 35%, 40%, 45%, 50%, 55%, 60%, 65%, 70%, 75%, 80%, 85%, 90%, 95%, 100%, or any value or range therein). In some embodiments, the non-myocytes may comprise fibroblasts (FBs), ECs, and MSCs in a ratio of about 4:2:1 of FBs:ECs:MSCs.

In some embodiments, the 3D myocardial infarct organoid comprises an apoptotic region that is due to lack of oxygen and is surrounded by a viable periphery comprising a region of about 20 μm to about 75 μm from the organoid edge, wherein the organoid edge is defined by the outermost DAPI stained nuclei.

In some embodiments, an organoid cross-section taken from the apoptotic region of the 3D myocardial infarct organoid comprises a ratio of a TUNEL-positive area to a DAPI-positive area may range from about 0.03 to about 1, about 0.1 to about 0.9, about 0.25 to about 0.75, or about 0.4 to about 0.6, wherein the ratio of the TUNEL-positive area to the DAPI-positive area of a region in a 3D cardiac organoid having no apoptotic region (e.g., control) is typically in a range from about 0 to about less than 0.03 (or less than about 0.01, 0.02, or about 0.025).

In some embodiments, the 3D myocardial infarct organoid may comprise a contraction amplitude from about 0% to about 5%, about 0% to about 4%, about 0% to about 3%, or from about 0% to about 4% (e.g., about 1%, about 2%, about 3% about 4%, or about 5%), wherein the contraction amplitude of a 3D cardiac organoid having no apoptotic region (e.g., control) is typically in a range of about 0.5% to about 10%, about 6% to about 10%, or about 8% to about 10%.

In some embodiments, the 3D myocardial infarct organoid may comprise a beat rate of about 0 to about 90 beats per minute, about 0 to about 50, about 0 to about 40, about 0 to about 30, about 0 to about 20, or about 0 to about 10 (e.g., about 0, 1, 2, 3, 4, 5, 6, 7, 8, 9, 10, 15, 20, 25, 30, 35, 40, 45, 50, 55, 60, 65, 70, 75, 80, 85, or about 90 beats per minute), wherein the beat rate of a 3D cardiac organoid having no apoptotic region (e.g., control) comprises a beat rate in a range from about 15 to about 75 beats per minute, about 55 to about 75 beats per minute, or about 60 to about 75 beats per minute.

In some embodiments, the 3D myocardial infarct organoid may comprise a calcium transient amplitude measured as a change in fluorescence divided by the starting fluorescence of about 0% to about 40%, about 0% to about 30%, about 0% to about 20%, or from about 0% to about 10% (e.g., about 0%, 1%, 2%, 3%, 4%, 5%, 6%, 7%, 8%, 9%, 10%, 15%, 20%, 25%, 30%, 35%, or about 40%), wherein the calcium transient amplitude of a 3D cardiac organoid having no apoptotic region (e.g., control) ranges from about 10% to about 100%, about 45% to about 100%, 55% to about 100%, about 65% to about 100%, about 75% to about 100%, or from about 85% to about 100%.

In some embodiments, the 3D myocardial infarct organoid may comprise upregulated or downregulated fibrosis-related genes, wherein the downregulated genes comprise, consists essentially of, or consists of COL22A1, COL11A2, FGF12, SPINT1, COL9A2, MMP13, HPN, CTSS, FGF7, A2M, COL9A1, FBN2, FGF9, LAMA3, FLT1, SMOC2, COL2A1, FGF2, LAMB3, LAMAS, PDGFB, SMAD3, ICAM5, LAMB2, FGF18, MMP9, CXCL12, COL19A1, FGF13, COL4A5, COL26A1, F11R, COL14A1, COL9A3, FGF1, ICAM1, HBEGF, MDK, ITGA6, TGFB3, LAMA2, RHOQ, RND2, TGFB2, LAMC2, CCDC88A, ITGAE, JUP, ITGAM, COL4A2, CDH2, ITGB2, TGFBR3, BTG1, COL4A1, COL4A3, PDGFRA, FGFR4, SDC4, MMPI, FGF6, ITGA8, and/or COL4A4,

and upregulated genes comprise, consists essentially of, or consists of FGF8, ITGB1BP1, ITGA7, TGIF2, MMP12, PIK3CA, RHOH, COL18A1, ITGAL, LAMC1, RPS6KB1, TGFBR2, RHOD, PIK3CD, MMP3, RAC1, MMP8, ITGB4, ARPC5L, ITGA3, COL17A1, ADAM9, CD2AP, KDR, LAMB1, COL12A1, ITGAX, ABIl, MMP15, FGF14, TGFB1I1, SDC3, ITGAV, FGFR1, TNC, FGF11, FGFR3, RHOJ, LAMA4, FBLN1, CTSL, DDR2, PDGFRB, MMP24, CD151, ACAN, RHOU, ARF4, COL3A1, FGFR2, COL7A1, ADAM15, CD47, COL10A1, VTN, RHOG, CAPN2, BGN, CXCR4, HTRA1, ICAM2, JAM3, ANG, TGIF1, ITGB7, CD63, RHOA, RHOC, ITGA2, DPP4, COL6A3, COL15A1, SDC2, SPOCK2, DCN, BCAN, COL13A1, ITGB1, MATN3, CLDN1, TIMP2, ARPC5, FN1, CST3, TPM3, MATN1, CD44, HAPLN1, SERPINH1, TIMP3, ITGB3, PLOD3, L1CAM, COL11A1, SPARC, COL6A2, FGF10, P4HA1, IBSP, GREM1, COL6A1, HAS1, CTGF, BMP1, RHOB, VCAN, TGFBR1, MMP10, COL5A2, MFAP2, FGF5, DPT, COL8A1, ITGB5, BDKRB1, COL1A2, TGFB1, MMP11, SERPINE1, LOXL2, FSCN1, SPP1, ITGA4, POSTN, COL5A1, RELN, MMP16, CCDC80, LAMA1, COL1A1, FBN1, ITGA5, LOX, MMP17, LOXL1, LCP1, SDC1, MMP2, MMP14, FAP, TNXB, TGFBI, HAS2, MFAP5, and/or CTSK.

In some embodiments, the 3D myocardial infarct organoid may comprise upregulated or downregulated calcium signaling-related genes comprising genes from the Kyoto Encyclopedia of Genes and Genomes (KEGG) calcium signaling pathway, wherein the downregulated genes comprise, consists essentially of, or consists of CACNA1G, EDNRB, CHRM1, ADRB1, PLCG2, ERBB4, RYR3, ERBB3, ATP2A2, ADRB2, P2RX7, PLCB1, ATP2A1, CAMK2A, RYR2, HRH2, PHKA1, PHKG1, ATP2B2, PDE1C, HTR4, CACNA1C, CAMK2B, SLC8A1, SLC25A5, CACNA1S, P2RX1, TBXA2R, CAMK2D, PRKACA, PHKA2, GRIN2C, PPIF, ADCY9, PTK2B, VDAC3, EGFR, VDAC2, PHKB, NOS2, PLCD1, GRIN2A, CALML4, P2RX6, TNNC2, VDAC1, PHKG2, CHRNA7, PRKCB, GRPR, SLC25A4, NOS1, CCKBR, ADORA2A, ADCY3, NTSR1, GRIN1, ADRA1A, PDGFRA, PPP3CB, NOS3, HTR2C, MYLK, TNNC1, and/or PLCG,

and the upregulated genes comprise, consists essentially of, or consists of GNA15, CACNA1H, GNAS, HTR5A, PTGFR, PTGER1, TACR1, RYR1, PRKACB, CCKAR, CD38, PTAFR, CALM2, PDE1A, PPP3R1, LHCGR, ADCY2, TACR2, PLCB3, GNA11, BDKRB2, PRKCG, STIM1, ADCY4, ATP2A3, GNA14, AVPR1A, CACNA1B, ITPR2, PPP3CC, HTR7, HTR2B, PPP3CA, PDGFRB, SPHK2, PRKCA, GRIN2D, PDE1B, GNAQ, CALM1, ITPKB, HRH1, CAMK4, P2RX4, PTGER3, ITPR1, ADCY7, ADORA2B, F2R, CACNA1E, BDKRB1, SPHK1, CACNA1A, ADRA1B, ADRB3, ITPR3, and/or ADCY8.

In some embodiments, the 3D myocardial infarct organoid comprises an elastic modulus of about 3 kPa to about 5 kPa, about 3.6 kPa to about 5 kPa, about 4 kPa to about 5 kPa, or about 4.5 kPa to about 5 kPa (e.g., about 3.5, 3.6, 3.7, 3.8, 3.9, 4.0, 4.1, 4.2, 4.3, 4.4, 4.5, 4.6, 4.7, 4.8, 4.9, or about 5 kPa), wherein the elastic modulus of a 3D cardiac organoid having no apoptotic region (e.g., control) ranges from about 2 kPa to less than 3.5 kPa, about 2 kPa to about 3 kPa, or from about 2 kPa to about 2.5 kPa (e.g., about 2, 2.1, 2.2, 2.3, 2.4, 2.5, 2.6, 2.7, 2.8, 2.9, 3.0, 3.1, 3.2, 3.3, or about 3.4 kPa).

In some embodiments, a 3D myocardial infarct organoid of the invention may beat asynchronously. In some embodiments, a 3D myocardial infarct organoid of the invention may beat synchronously. In some embodiments, when synchrony of beat is measured in a population of 3D myocardial infarct organoids of the invention, all of the organoids in the population may beat synchronously. In some embodiments, when synchrony of beat is measured in a population of 3D myocardial infarct organoids of the invention, all of the organoids in the population may beat asynchronously. In some embodiments, when synchrony of beat is measured in a population of 3D myocardial infarct organoids of the invention, some of the organoids in the population may beat synchronously and others in the population may beat asynchronously. Thus, in some embodiments, a population of 3D myocardial infarct organoids may comprise a subpopulation of organoids that beat asynchronously. In some embodiments, a population of 3D myocardial infarct organoids of the invention may have a beat asynchrony of about 30% to 100% (e.g., about 30, 35, 40, 45, 50, 55, 60, 65, 70, 75, 80, 85, 90, 95 or 100% of the organoids in a population of 3D myocardial infarct organoids of the invention may beat asynchronously).

The 3D myocardial infarct organoid of the invention may comprise any one or more of the above described features in any combination thereof.

II. Methods of Making a Three Dimensional (3D) Myocardial Infarct Organoid and/or a 3D Myocardial Ischemia-Reperfused Organoid

One aspect of the invention relates to a method of making a 3D myocardial infarct organoid, the method comprising culturing cardiomyocytes with non-myocytes for about 1 to about 20 days to form a self-assembled 3D cardiac organoid under normoxic conditions and exposing the 3D cardiac organoid to hypoxic conditions for about 1 to about 20 days, thereby generating the 3D myocardial infarct organoid.

In some embodiments, the cardiomyocytes include, but are not limited to, induced pluripotent stem cell-derived cardiomyocytes (iPSC-CMs), cardiac progenitor cells, primary cardiomyocytes, or any combination thereof. In some embodiments, non-myocytes include, but are not be limited to, fibroblasts (FBs), endothelial cells (ECs), and mesenchymal cells (MSCs). Exemplary endothelial cells include but are not limited to human umbilical vein endothelial cells (HUVECs). Exemplary mesenchymal cells (MSCs) include but are not limited to human adipose derived stem cells (hADSCs).

In some embodiments, the cardiomyocytes and/or non-myocytes are from a mammal. A mammal may be a human, a nonhuman primate, a domesticated mammal (e.g., a dog, a cat, a rabbit, a guinea pig, a rat), or a livestock and/or agricultural mammal (e.g., a horse, a bovine, a pig, a goat). In some embodiments, the mammal is a human. In some embodiments, the cardiomyocytes and/or myocytes are from a human. In some embodiments, the cardiomyocytes and myocytes may be from a subject (e.g., human) undergoing therapy or in need of therapy for a cardiac disease. In such cases, the organoids developed from these cells may be used for development of a personalized therapeutic protocol for the subject.

In some embodiments, the cardiomyocytes may be cultured with the non-myocytes at a ratio of about 95:5 to about 5:95, about 90:10 to about 10:90, about 85:15 to about 15:85, about 70:30 to about 30:70, or about 60:40 to about 40:60 of cardiomyocytes to non-myocytes (e.g., about 98:2, about 95:5, about 90:10, about 85:15, about 80:20, about 75:25, about 70:30, about 65:35, about 60:40, about 55:45, about 50:50, about 45:55, about 40:60, about 35:65, about 30:70, about 25:75, about 20:80, about 15:85, about 10:90, about 5:95, or about 2:98 of cardiomyocytes to non-myocytes.

In some embodiments, the amount of fibroblasts (FBs), endothelial cells (ECs), and mesenchymal cells (MSCs) comprising the total amount of non-myocytes that are being cultured with the cardiomyocytes can vary. For example, in some embodiments, the non-myocytes cultured with the cardiomyocytes may comprise FBs in amount of about 1% to about 100%, about 20% to about 80%, about 40% to about 70%, or about 50% to about 60% based on the total number of non-myocytes (e.g., about 1%, 2.5%, 5%, 10%, 15%, 20%, 25%, 30%, 35%, 40%, 45%, 50%, 55%, 60%, 65%, 70%, 75%, 80%, 85%, 90%, 95%, 100%, or any value or range therein). In some embodiments, the non-myocytes cultured with the cardiomyocytes may comprise ECs in an amount of about 1% to about 100%, about 10% to about 80%, about 20% to about 50%, or about 25% to about 35% based on the total number of non-myocytes (e.g., about 1%, 2.5%, 5%, 10%, 15%, 20%, 25%, 30%, 35%, 40%, 45%, 50%, 55%, 60%, 65%, 70%, 75%, 80%, 85%, 90%, 95%, 100%, or any value or range therein). In some embodiments, the non-myocytes cultured with the cardiomyocytes comprise MSCs in an amount of about 1% to about 100%, about 5% to about 50%, or about 10% to about 20% based on the total number of non-myocytes (e.g., about 1%, 2.5%, 5%, 10%, 15%, 20%, 25%, 30%, 35%, 40%, 45%, 50%, 55%, 60%, 65%, 70%, 75%, 80%, 85%, 90%, 95%, 100%, or any value or range therein). In some embodiments, the non-myocytes cultured with the cardiomyocytes comprise FBs, ECs, and MSCs in a ratio of about 4:2:1 of FBs:ECs:MSCs.

In some embodiments, the cardiomyocytes (e.g., iPSC-CMs) may be cultured with the non-myocytes (e.g., FBs, endothelial cells, and/or mesenchymal stem cells) at a total concentration of about 1×10⁵ cells/mL to about 1×10⁷ cells/mL (e.g., about 1×10⁵, 2×10⁵, 3×10⁵, 4×10⁵, 5×10⁵, 5×10⁵ 6×10⁵, 7×10⁵, 8×10⁵,9×10⁵, 1×10⁶, 2×10⁶, 3×10⁶, 4×10⁶, 5×10⁶, 5×10⁶ 6×10⁶, 7×10⁶, 8×10⁶, 9×10⁶, 1×10⁷ cells/mL, or any value or range therein).

In some embodiments, the cardiomyocytes may be cultured with the non-myocytes in a cell suspension in microwells composed of non-fouling materials. The cell suspension may comprise one or more culture media suitable for culturing cardiomyocytes and/or non-myocytes. Culture media for culturing cardiomyocytes and/or non-myocytes are well known in the art. The type of culture media in a cell suspension can vary. For example, a cell suspension may comprise a larger amount of cardiomyocyte cell culture media when the amount of cardiomyocytes being cultured is greater than the amount of non-myocytes. In another example, the cell suspension may comprise a larger amount of non-myocyte cell culture media when the amount of non-myocytes being cultured is greater than the amount of cardiomyocytes being cultured in the cell suspension. Thus, the amounts of all the specific media may be ratiometric reflecting the cell ratio of the organoid.

The micro-wells employed in the inventive method can be any micro-wells comprising non-fouling materials known in the art that are suitable for microtissue fabrication. In some embodiments, the non-fouling materials comprise agarose or non-adhesive self-assembly plates, such as the InSphero Gravity TRAP ultra-low attachement plate. The non-fouling materials may comprise any suitable material, such as, for example, agarose gel, polyethylene glycol, alginate, hyaluronic acid, polyacryylic acid, polyacrylic amide, polyvinyl alcohol, polyhydroxyethyl methacrylate, methacrylated dextrans, poly(N-isopropylacrylamide), and any combination thereof. In some embodiments, the substrate may be any suitable unfouling hydrogel.

In some embodiments, the cardiomyocytes are cultured with the non-myocytes for about 1 to about 20 days, about 5 to about 15 days, or about 8 to about 12 days (e.g., about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, and any range or value herein).

In some embodiments, the cardiomyocytes are cultured with the non-myocyte cells thereby forming a self-assembled 3D cardiac organoid under normoxic conditions, wherein normoxic conditions comprise a partial pressure of oxygen in the gas phase of about 16% to about 20.9% of the total barometric pressure (or at least about 16%, about 17%, about 18%, about 19%, or at least about 20% of the total barometric pressure).

In some embodiments, the 3D cardiac organoid is exposed to hypoxic conditions, wherein hypoxic conditions comprise a partial pressure of oxygen in the gas phase of less than about 15%, about 14%, about 13%, about 12%, about 11%, about 10%, about 9%, about 8%, about 7%, about 6%, about 5%, about 4%, about 3%, about 2%, or at least lower than about 1% of the total barometric pressure. In some embodiments, the hypoxic condition can include 0% oxygen of the total barometric pressure.

In some embodiments, the 3D myocardial organoid is exposed to the hypoxic conditions for 1 to about 20 days, about 5 to about 15 days, or about 8 to about 12 days (e.g., about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, or about 20 days, and any range or value therein).

In some embodiments, the cardiomyocytes are cultured with the non-myocytes in the presence of an additional agent selected from norepinephrine, angiotensin II, TNF-alpha, interfering RNAs, microRNAs, matrix metalloproteases, and any combination thereof. The amount of the additional agent can vary. For example, in some embodiments, the amount of the additional agent may range from about 0.01 μM to about 10 μM, about 1 μM to about 8 μM, or from about 3 μM to about 5 μM (e.g., about 1 μM, about 2 μM, about 3 μM, about 4 μM, about 5 μM, about 6 μM, about 7 μM, about 8 μM, about 9 μM, or from about 10 μM, or any range or value therein).

Another aspect of the invention relates to a method of making a 3D myocardial ischemia-reperfused organoid, wherein the 3D myocardial infarct organoid of the invention is exposed to normoxic conditions. For example, in some embodiments, a method of making a 3D myocardial ischemia-reperfused organoid may comprise the steps of culturing cardiomyocytes with non-myocytes for about 1 to about 20 days to form a 3D cardiac organoid under normoxic conditions, exposing the 3D cardiac organoid under hypoxic conditions for about 1 day to 20 days to form the 3D myocardial infacrt organoid of the invention, which is exposed to normoxic conditions again and/or exposed to/contacted with fresh culture media for about 5 seconds to about 20 days. In some embodiments, the normoxic conditions employed in the exposure of the 3D myocardial cardiac organoid of the invention comprises a partial pressure of oxygen in the gas phase of about 16% to about 20.9% of the total barometric pressure (or at least about 16%, about 17%, about 18%, about 19%, about 20% of the total barometric pressure, or any range or value therein).

In some embodiments, the 3D myocardial infarct organoid may be exposed to the normoxic conditions for about 5 seconds to about 20 days, about lday to about 15 days, or about 8 days to about 12 days (e.g., about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, or any range or value therein).

In some embodiments, the 3D myocardial infarct organoid may be exposed to the normoxic conditions for about 5 seconds to about 1 day, about 1 minute to about 1 day, about 2 minutes to about 1 day, about 5 minutes to about 1 day, about 10 minutes to about 1 day, about 20 minutes to about 1 day, about 30 minutes to about 1 day, about 40 minutes to about 1 day, about 50 minutes to about 1 day, about 1 hour to about 1 day, about 10 minutes to about 1 hour, about 30 minutes to about 1 hour, about 1 hour to about 2 hours, about 1 hour to about 12 hours, about 6 hours to about 10 hours (e.g., about 5 sec, 1 min., 5 min., 10 min., 20 min., 30 min., 1 hour, 2 hours, 3 hours, 4 hours, 5 hours, 6 hours, 7 hours, 8 hours, 9 hours, 10 hours, 11 hours, 12 hours, 14 hours, 16 hours, 18 hours, 20 hours, 22 hours, 24 hours, or any range or value therein).

In some embodiments, a 3D myocardial infarct organoid may be contacted with fresh culture media, thereby exposing the 3D myocardial infarct organoid to the oxygen present in the fresh culture media and generating a 3D myocardial ischemia-reperfused organoid. In some embodiments, fresh culture media may be added to the culture medium of a 3D myocardial infarct organoid in addition to exposing the 3D myocardial infarct organoid to normoxic conditions (e.g., a partial pressure of oxygen in the gas phase of about 16% to about 20.9% of the total barometric pressure) to generate a 3D myocardial ischemia-reperfused organoid. The amount of fresh culture media added may vary.

A 3D myocardial infarct organoid and/or a 3D myocardial ischemia-reperfused organoid can be in any suitable shape. For example, in some embodiments, the 3D myocardial infarct organoid and/or the 3D myocardial ischemia-reperfused organoid can be in the shape of a spheroid. In some embodiments, the spheroid comprises an average diameter of about 100 to about 1000 μpm, about 200 to about 800 μm, or about 200 to about 400 μm (or of about 100 μm, about 150 μm, about 200 μm, about 250 μm, about 300 μm, about 350 μm, about 400 μm, about 450 μm, about 500 μm, about 550 μm, about 600 μm, about 650 μm, about 700 μm, about 750 μm, about 800 μm, about 850 μm, about 900 μm, about 950 μm, about 1000 μm, or any value or range therein).

III. Method of Using a Three Dimensional (3D) Myocardial Infarct Organoid and/or a 3D Myocardial Ischemia-Reperfused Organoid

An aspect of the invention relates to employing a 3D myocardial infarct organoid of the invention and/or a 3D myocardial ischemia-reperfused organoid of the invention in a method of screening a compound for its ability to improve or diminish cardiac function. The ability of the compound to improve or diminish cardiac function is determined by contacting the 3D myocardial infarct organoid of the invention and/or the 3D myocardial ischemia-reperfused organoid of the invention with a compound followed by measuring one or more characteristics of the organoid that reflect modulation of cardiac function (e.g., size of the interior apoptotic region of the 3D myocardial infarct organoid and/or 3D myocardial ischemia-reperfused organoid, ratio of the TUNEL-positive area to the DAPI-positive area in the apoptotic region, contraction amplitude, beat rate, calcium transient amplitude, and/or elastic modulus). The measurements of these characteristics can then be compared with corresponding reference values for a 3D myocardial infarct organoid of the invention and/or a 3D myocardial ischemia-reperfused organoid of the invention that has not been contacted with the compound, thereby determining the effect(s) of the compound on one or more of the measured characteristics that reflect cardiac function. The compound can be any compound of interest, such as, for example, a therapeutic compound. Exemplary therapeutic compounds include, but are not limited to, a therapeutic compound for treating cardiovascular disease, diabetes, kidney disease, liver disease, and/or cancer. In some embodiments, the compound is a small-molecule, nucleic-acid based drug and/or protein-based drug.

In some embodiments, a method of screening a compound for improving cardiac function may comprise contacting the 3D myocardial infarct organoid of the invention or the 3D myocardial ischemia-reperfused organoid of the invention with the compound and measuring in the 3D myocardial infarct organoid or 3D myocardial ischemia-reperfused organoid one or more of : (a) size of the interior apoptotic region, (b) ratio of a TUNEL-positive area to a DAPI-positive area in the apoptotic region, (c) contraction amplitude, (d) beat rate, (e) calcium transient amplitude, and/or (0 elastic modulus.

In some embodiments, a compound may be determined to improve cardiac function when the size of the interior apoptotic region is reduced by at least about 30%, about 40%, about 50%, about 60%, about 70%, about 80%, about 90%, or about 100% when compared to a control 3D myocardial infarct organoid and/or a 3D myocardial ischemia-reperfused organoid that has not been contacted with the compound. The size of the apoptotic region can vary but typically ranges from about 20 ₗam to about 75 ₗam in a control 3D myocardial infarct organoid and/or control 3D myocardial ischemia-reperfused organoid that has not been contacted with the compound.

In some embodiments, a compound may be determined to improve cardiac function when the ratio of TUNEL-positive area to DAPI-positive area is reduced by at least about 30%, about 40%, about 50%, about 60%, about 70%, about 80%, about 90%, or about 100% when compared to a control 3D myocardial infarct organoid and/or control 3D myocardial ischemia-reperfused organoid that has not been contacted with the compound. This ratio can vary but typically ranges from about 0.03 to about 1 in a control 3D myocardial infarct organoid and/or control 3D myocardial ischemia-reperfused organoid that has not been contacted with the compound.

In some embodiments, a compound may be determined to improve cardiac function when the contraction amplitude is increased by at least about 30%, about 40%, about 50%, about 60%, about 70%, about 80%, about 90%, or about 100% when compared to a control 3D myocardial infarct organoid and/or control 3D myocardial ischemia-reperfused organoid that has not contacted with the compound. This contraction amplitude can vary but typically ranges from about 0% to about 5% in a control 3D myocardial infarct organoid and/or control 3D myocardial ischemia-reperfused organoid that has not contacted with the compound.

In some embodiments, a compound may be determined to improve cardiac function when the calcium transient amplitude is increased by at least about 30%, about 40%, about 50%, about 60%, about 70%, about 80%, about 90%, or about 100% when compared to a control 3D myocardial infarct organoid and/or control 3D myocardial ischemia-reperfused organoid that has not been contacted with the compound. This calcium transient amplitude can vary but typically ranges from about 0% to about 40% in a control 3D myocardial infarct organoid and/or control 3D myocardial ischemia-reperfused organoid that has not been contacted with the compound.

In some embodiments, a compound may be determined to improve cardiac function when the elastic modulus is decreased by at least about 30%, about 40%, about 50%, about 60%, about 70%, about 80%, about 90%, or about 100% when compared to a control 3D myocardial infarct organoid and/or control 3D myocardial ischemia-reperfused organoid that has not been contacted with the compound. The elastic modules of a control 3D myocardial infarct organoid and/or a control 3D myocardial ischemia-reperfused organoid can vary but typically ranges from about 3 kPa to about 5 kPa.

Another aspect of the invention relates to a method for screening a compound for diminishing cardiac function. For example, such compounds include therapeutic compounds used for treating diseases other than cardiovascular diseases. Screening of any cardiovascular effects of such compounds in a 3D myocardial infarct organoids and/or ischemic-reperfused myocardial organoid of the inventions provides useful information as to the potential cardiotoxicity associated with these compounds when administered to a mammals (e.g., a human) that is already cardio-compromised (i.e., wherein the heart is not functioning at full capacity). In some embodiments, the method may comprise contacting the 3D myocardial infarct organoid of the invention or the 3D myocardial ischemia-reperfused organoid of the invention with a test compound and measuring one or more of: (a) size of the interior apoptotic region, (b) ratio of a TUNEL-positive area to a DAPI-positive area in the apoptotic region, (c) contraction amplitude, (d) beat rate, (e) calcium transient amplitude, and/or (f) elastic modulus.

In some embodiments, a compound may be contact with a population of 3D myocardial infarct organoids or a population of 3D ischemia-reperfused organoids. In some embodiments, a population of 3D myocardial infarct organoids or a population of 3D ischemia-reperfused organoids may comprise about 2 to about 100, about 2 to about 80, about 2 to about 70, about 2 to about 50, about 2 to about 40, about 2 to about 35, about 2 to about 25 or about 2 to about 10 3D myocardial infarct organoids or 3D ischemia-reperfused organoids. In some embodiments, the number (percentage of the total population) of asynchronously beating 3D myocardial infarct organoids or 3D ischemia-reperfused organoids in the population may be determined after contacting the population with a test compound. In some embodiments, a compound may be determined to improve cardiac function when the percentage of asynchronously-beating organoids in the population (e.g., an asynchronously-beating subpopulation) is decreased by more than about 30%, about 40%, about 50% about 60%, about 70%, about 80%, about 90%, or about 100% when compared to a control 3D myocardial infarct organoid and/or control 3D myocardial ischemia-reperfused organoid that has not been contacted with the compound. In a control population of 3D myocardial infarct organoids and/or a control population of 3D myocardial ischemia-reperfused organoids that have not been contacted with the compound, the percentage of organoids that make up the asynchronously-beating subpopulation may vary but typically ranges from about 30% to about 100%.

In some embodiments, a compound may be determined to diminish cardiac function when the size of the interior apoptotic region is increased by at least about 30% about 40% about 50%, about 60%, about 70%, about 80%, about 90%, or about 100% when compared to a control 3D myocardial infarct organoid and/or control 3D myocardial ischemia-reperfused organoid that is not contacted with the compound. This size of the apoptotic region can vary but typically ranges from about 20 μm to about 75 μm in a control 3D myocardial infarct organoid and/or a control 3D myocardial ischemia-reperfused organoid that has not been contacted with the compound.

In some embodiments, a compound may be determined to diminish cardiac function when the ratio of TUNEL-positive area to DAPI-positive area is increased by at least 30%, about 40%, about 50%, about 60%, about 70%, about 80%, about 90%, or about 100% when compared to a control 3D myocardial infarct organoid and/or control 3D myocardial ischemia-reperfused organoid that has not been contacted with the compound. This ratio can vary but typically ranges from about 0.03 to about 1 in a control 3D myocardial infarct organoid and/or a control 3D myocardial ischemia-reperfused organoid that has not been contacted with the compound.

In some embodiments, a compound may be determined to diminish cardiac function when the contraction amplitude is decreased by at least about 30%, about 40%, about 50%, about 60%, about 70%, about 80%, about 90%, or about 100% when compared to a control 3D myocardial infarct organoid and/or control 3D myocardial ischemia-reperfused organoid that has not been contacted with the compound. This contraction amplitude can vary but typically ranges from about 0% to about 5% in a control 3D myocardial infarct organoid and/or a control 3D myocardial ischemia-reperfused organoid that has not been contacted with the compound.

In some embodiments, a compound may be determined to diminish cardiac function when the calcium transient amplitude is decreased by at least about 30%, about 40%, about 50%, about 60%, about 70%, about 80%, about 90%, or about 100% when compared to a control 3D myocardial infarct organoid and/or control 3D myocardial ischemia-reperfused organoid that has not been contacted with the compound. This calcium transient amplitude can vary but typically range from about 0% to about 40% in a control 3D myocardial infarct organoid and/or a control 3D myocardial ischemia-reperfused organoid that has not been contacted with the compound.

In some embodiments, a compound may be determined to diminish cardiac function when the elastic modulus is increased by about 30%, about 40%, about 50%, about 60%, about 70% about 80%, about 90%, or about 100% when compared to a control 3D myocardial infarct organoid and/or control 3D myocardial ischemia-reperfused organoid that has not contacted with the compound. This elastic modulus of a control 3D myocardial infarct organoid and/or a control 3D myocardial ischemia-reperfused organoid can vary but typically ranges from about 3 kPa to about 5 kPa.

In some embodiments, a compound may be contact with a population of 3D myocardial infarct organoids or a population of 3D ischemia-reperfused organoids. In some embodiments, a population of 3D myocardial infarct organoids or a population of 3D ischemia-reperfused organoids may comprise about 2 to about 100, about 2 to about 80, about 2 to about 70, about 2 to about 50, about 2 to about 40, about 2 to about 35, about 2 to about 25 or about 2 to about 10 3D myocardial infarct organoids or 3D ischemia-reperfused organoids. In some embodiments, the number (percentage of the total population) of asynchronously beating 3D myocardial infarct organoids or 3D ischemia-reperfused organoids in the population may be determined after contacting the population with a test compound. In some embodiments, a compound may be determined to diminish cardiac function when the percentage of asynchronously-beating organoids in the population (e.g., an asynchronously-beating subpopulation) is decreased by more than about 30%, about 40%, about 50% about 60%, about 70%, about 80%, about 90%, or about 100% when compared to a control 3D myocardial infarct organoid and/or control 3D myocardial ischemia-reperfused organoid that has not been contacted with the compound. In a control population of 3D myocardial infarct organoids and/or a control population of 3D myocardial ischemia-reperfused organoids that have not been contacted with the compound, the percentage of organoids that make up the asynchronously-beating subpopulation may vary but typically ranges from about 30% to about 100%.

The invention will now be described with reference to the following examples. It should be appreciated that these examples are not intended to limit the scope of the claims to the invention, but are rather intended to be exemplary of certain embodiments. Any variations in the exemplified methods that occur to the skilled artisan are intended to fall within the scope of the invention.

### EXAMPLES

### Example 1

### Oxygen Diffusion Mathematical Modeling

A computational finite element model of nutrient diffusion and transport was developed to predict oxygen concentration profiles within cardiac spheroids (cardiomyocyte-only) based on Fick's Second Law of Diffusion. Given the developmental similarities between hiPSC-CMs and neonatal rat cardiomyocytes¹, neonatal cardiomyocyte metabolic data was used according to the detailed oxygen consumption work of Brown and others.² Specifically, the oxygen diffusivity value (D_{Ox}=3.0×10⁻⁶ cm²/s) and cardiomyocyte specific consumption rate constants for oxygen (Vₘₐₓ=5.44×10⁻⁸ nmol/cell/s and Kₘ=3.79 nmol/ml) were derived from Brown and others.² Next, the concentration-dependent nutrient consumption rate of the cardiomyocytes was modeled by the Michaelis-Menten equation R=ρ_{c}Vₘₐₓ[C]/Kₘ+[C], where ρ_{c} represents spheroid cell density, Vₘₐₓ maximum rate at high substrate concentration, and Kₘ Michaelis-Menten constant. Physiological (20%) and hypoxic (10%) culture conditions were accounted for in the boundary conditions of the model. In a spherical coordinate system, the internal oxygen concentration profile is governed by the equation D/r²∂/∂ᵣ(r²∂C/∂ᵣ)−R=0, where C represents nutrient concentration, r radial distance from spheroid center, D nutrient diffusivity, and R nutrient consumption rate. Upon compiling all of the relevant equations, the finite element model was numerically solved by the software COMSOL Multiphysics, from which the internal oxygen concentration profiles were determined in simulated cardiomyocyte spheroids. In evaluating the finite element model, the semi-circular concentration profiles obtained by solving the finite element model on COMSOL were reformatted into line graphs that showed the change in oxygen levels based on radial position from the spheroid center. These plots were then assessed for trends that indicated the effects of external oxygen concentration on the internal oxygen distributions within the cardiac spheroid.

### Example 2

### Cell Culture Protocol

Human induced pluripotent stem cell-derived cardiomyocytes (hiPSC-CMs) (iCell Cardiomyocytes, Cellular Dynamics International-CDI, Madison, Wis., USA) were cultured according to the manufacturer's protocol. iCell Cardiomyocytes iPSC donor 01434 (CDI) were used for all experiments and iCell Cardiomyocytes iPSC donor 11713 were used where notated. Briefly, hiPSC-derived cardiomyocytes were plated on 0.1% gelatin coated 6-well plates in iCell Cardiomyocyte Plating Medium (CDI) at a density of about 3×10⁵ to 4.0×10⁵ cells/well and incubated at 37° C. in about 5% CO₂ for about 4 days. Two days after plating, the plating medium was removed and replaced with 4 mL of iCell Cardiomyocytes Maintenance Medium (CDI). After 4 days of monolayer pre-culture, cells were detached using trypLE Express (Gibco Life Technologies, Grand Island, N.Y.) and prepared for spheroid/organoid fabrication. Human cardiac ventricular fibroblasts (FBs) (Lot#: 401462, Lonza, Basel, Switzerland) were cultured in FGM-2 media (Lonza) were used at passage 3-5 for spheroid/organoid fabrication. Human umbilical vein endothelial cells (HUVECs) (Lot#: 471466, Lonza) were cultured in EGM-3 media (Lonza) and were used at passage 2-4 for organoid fabrication. Human adipose-derived stem cells (hADSCs) (Lot#: 410257, Lonza) were cultured in low glucose Dulbecco's modified Eagle's medium with 10% fetal bovine serum (FBS) and 1% penicillin-streptomycin, 1% glutamine and 1% antimycin (Gibco Life Technologies, Grand Island, N.Y.). hADSCs were used at passage 5-7 for organoid fabrication.

### Example 3

### Organoid and Spheroid Fabrication Protocol

Non-adhesive agarose hydrogel molds were used as microtissue fabrication molds made from commercial master micro-molds from Microtissues, Inc (Providence, R.I.). Working cell suspensions of each cell type were used at about 4.0×10⁶ cells/ml to make organoid cell ratio mixtures of about 50% hiPSC-CMs and about 50% non-myocyte (4:2:1 ratio of FBs, HUVECs, hADSCs, respectively) and mixed with 1 volume media for a final concentration of about 2.0×10⁶ cells/ml. hiPSC-CM only spheroids were fabricated using 100% hiPSC-CMs at a final concentration of about 2.0×10⁶ cells/ml. Approximately 75 μl of the cell suspension was pipetted into each agarose mold. After the cells settled into the recesses of the mold (15 min), additional media was added to submerge the molds in a 12-well plate and exchanged about every 2 days for the length of the experiment (about 10 days). Day 0 (D0) of the experiment was marked after about 4 days of spheroid assembly. Culture media for cardiac organoids was comprised of a ratiometric combination of cell-specific media reflecting the cell ratio of the organoid. In organoid media, CM-specific media (i.e., CDI hiPSC-CM media supplied without glucose) was substituted with glucose-containing F12/DMEM media with 10% FBS, 1% glutamine, and 1% non-essential amino acids (Gibco).

### Example 4

### Cardiac Infarction Organoid Protocol

For the cardiac infarction protocol, microtissues (about150 μm radius on D0) were placed in a hypoxia chamber within the incubator at about 10% O₂ with 1 μM of norepinephrine (NE, A7257, Sigma) after 4 days of pre-culture. Media was changed with fresh NE every 2 days for the length of the experiment (10 days). For extended validation studies, 20 ng/ml of human recombinant transforming growth factor beta 1 (TGF-β1, ab50036, Abcam, Cambridge, UK), 10 nM of JQ1 (SML1524, Sigma), and 2 ng/ml of human recombinant vascular endothelial growth factor (VEGF, CC-4114A, Lonza) was added to the media every 2 days for infarct organoids for the length of the experiment (10 days).

### Example 5

### Contraction Analysis of Beating Spheroids.

Videos of spontaneously beating spheroids from each group were recorded immediately out of the incubator (to reduce temperature induced changes in beating) for each condition using a Carl Zeiss Axiovert A1 Inverted Microscope and Zen 2011 software (Zeiss, Gottingen, Germany). Threshold edge-detecting in ImageJ software (NIH—US National Institutes of Health) was used on high contrast spheroid picture series and graphed to realize beating profiles of fractional area change, from which contraction amplitude was calculated. Contraction amplitudes were calculated as the percent change in fractional area change amplitude between contraction and relaxation. Beat rate was calculated as the number of beats per second.

### Example 6

### RNA-Sequencing Analysis

Total RNA was isolated one day after last media change (D11) according to the kit and protocol of an Omega bio-tek E.Z.N.A. Total RNA kit I (Omega bio-tek, Norcross, Ga.) with the addition of the Homogenizer Columns (Omega bio-tek) during the homogenization step for organoids. For each group, 30-35 organoids were used for RNA isolation. To prepare RNA-Seq libraries, the TruSeq RNA Sample Prep Kit (Illumina, San Diego, Calif., USA) was utilized; 100-200 ng of total input RNA was used in accordance with the manufacturer's protocol. High throughput sequencing (HTS) was performed using an Illumina HiSeq2000 with each mRNA library sequenced to a minimum depth of about50 million reads. A single end 50 cycle sequencing strategy was employed. Data were subjected to Illumina quality control (QC) procedures (>80% of the data yielded a Phred score of 30). RNA-Seq data has been submitted to the NCBI Gene Expression Omnibus, accession number GSE113871

Secondary analyses was carried out on an OnRamp Bioinformatics Genomics Research Platform as previously described (OnRamp Bioinformatics, San Diego, Calif., USA).⁴ OnRamp's Advanced Genomics Analysis Engine utilizes an automated RNA-Seq workflow to process data, including (1) FastQC to perform data validation and quality control; (2) CutAdapt⁵ to trim and filter adapter sequences, primers, poly-A tails and other unwanted sequences; (3) TopHat2⁶ to align mRNA-Seq reads to h19 human genome using the ultra-high-throughput short read aligner Bowtie2⁷; (4) HTSeq⁸ to establish counts which represent the number of reads for each transcript; and (5) DESeq2⁹ to perform DE analysis, which enabled the inference of differential signals with robust statistical power. Transcript count data from DESeq2 analysis of the samples were sorted according to their adjusted p-value (or q-value), which is the smallest false discovery rate (FDR) at which a transcript is called significant. FDR is the expected fraction of false positive tests among significant tests and was calculated using the Benjamin-Hochberg multiple testing adjustment procedure and set to q≤0.1. Advaita Bio's iPathwayGuide was used to perform further characterization, including differential expressed (DE) gene summary, gene ontology, and pathway analysis.¹⁹

### Example 7

### Transcriptional Comparative Analysis

Previously published transcriptomic datasets were obtained through the Gene Expression Omnibus (GEO). Microarray data from a large human heart failure study¹¹ (GSE5406, “nonfailing” and “ischemic” samples), a time-course mouse myocardial infarction study¹² (GSE775, “lv-control”, “MI_ilv-below MI ligation site”, and “MI_nilv-above MI ligation site” samples), and a time-course porcine myocardial infarction study¹³ (GSE34569, “sham-operated”, “infarct core”, and “remote” samples) were analyzed using the interactive GEO web tool (limma-based), GEO2R, to obtain summary files of genes ordered by significance.¹⁴⁻¹⁶ For Venn diagram comparison across datasets, differentially expressed (DE) genes (p<0.05) were directly compared for common genes using Venny and graphed using VennDis.^{17,18} DE genes for Venn diagrams were obtained from comparisons of control vs infarct (organoids), nonfailing vs ischemic failing (human), control vs MI infarct zone at 1 wk (mouse), and sham vs MI infarct at 1 wk (porcine). RNA-seq datasets were obtained from GEO from a public human heart failure studyl⁹ (GSE46224, “ischemic cardiomyopathy (ICM)” and “nonfailing” samples) and a mouse 2 wk myocardial infarction study²⁰ (GSE52313, “sham” and “MI” samples). Before merging gene lists for principal component analysis (PCA), organoid, human, and mouse RNA-seq data were normalized to the size of the library through the R package DESeq2 estimateSizeFactors function. For human RNA-seq data, starting counts were calculated based on the supplied RPKM and read counts/mapping details for nonfailing and ICM samples from the associated publication.¹⁹ After merging normalized organoid, human, and mouse RNA-seq data, the resulting 4,765 shared genes were loge-transformed followed by quantile normalized using the normalize. quantiles function in the preprocessCore package. PCA was then performed using the prcomp function in R and plotted as scatter plots (using jitter) with basic plotting tools in R. Principal component gene loadings were used as rankings for genes in gene set enrichment analysis (GSEA) with the gene ontology c5.all.v6.1.symbols.gmt file, run with default settings. GSEA terms were considered significant with a normalized p-value (NOM p-value) less than 0.05 and a false discovery rate (FDR) less than 0.25. Supplemental PCA using normalized mouse heart sham samples from another study (GSE96561) was performed after merging data into heart failure data, resulting in 4,244 shared genes.²¹

Given the lack of cardiac fibrosis pathway term and subjective nature of fibrosis in the heart, the fibrosis-related gene set was constructed based on the “extracellular matrix organization” GO term in addition to a “greedy”-based selection that incorporated common factors in fibrosis and (myo)fibroblast-related genes for a total of 349 genes. The calcium signaling-related gene set was defined as the genes contained in the “calcium signaling pathway” KEGG term 4020 for a total of 182 genes. Cardiac organoid RNA-seq and mouse 1 wk MI microarray (GSE775) were first intersected to isolate for common genes across platforms and then merged again with the filter gene sets, resulting in 208 fibrosis-related genes in organoids and mouse and 121 calcium-related genes in organoids and mouse. Heatmaps of fibrosis-related gene sets in organoids and mouse data were constructed separately using the pheatmap package in R with hierarchical clustering of samples (columns) with category-ordered genes (rows). Heatmaps of calcium handling-related gene sets in organoids and mouse data were constructed in like manner but with row order based on the organoid log-fold change.

### Example 8

### Fluorescent Imaging and Analysis

Freshly collected organoids were flash frozen in Tissue-Tek OCT compound (Sakura, Torrance, Calif.). Embedded spheroids were cryosectioned into 7 μm thickness layers onto glass slides for immunofluorescence staining. The sections were fixed with pre-cooled acetone (−20° C.) for 10 min. After washing (2 times at 5 min) in PBS with 0.1% Triton X-100 (PBST) (Sigma), blocking buffer was made with 10% serum corresponding to host species of secondary antibody in PBST and added to sections for 1 hr at room temperature. Sections were incubated with primary antibody diluted in PBST (1:200) overnight at 4° C. or 2 hrs at room temperature: mouse anti-alpha smooth muscle actin (A5228, Sigma), mouse anti-alpha sarcomeric actinin (ab9465, Abcam), rabbit anti-collagen type I (ab34710, Abcam), rabbit anti-vimentin (ab92547, Abcam), rabbit anti-von Willebrand factor (ab6994, Abcam). After washing in PBST (2 times at 5 min), sections were incubated with complement secondary antibodies or conjugated primary antibodies diluted in PBST for 1 hr at room temperature: Alexa Fluor 488 phalloidin (A12379, Thermo), goat anti-mouse Alexa Fluor 546 (A1103, Thermo), goat anti-rabbit Alexa Fluor 647 (111-605-144, Jackson ImmunoResearch, West Grove, Pa.). After washing in PBST (2 times at 5 min), nuclei were counterstained with DAPI (Molecular Probes/Invitrogen, Eugene, Oreg.) diluted in PBST for 15 min at room temperature. Following the final wash procedure (PBST, 2 times at 5 min), glass cover slips were added using Fluoro-Gel (Electron Microscopy Sciences, Hatfield, Pa.) and stored in 4° C. until imaging. TCS SP5 AOBS laser scanning confocal microscope (Leica Microsystems, Inc., Exton, Pa.) was for imaging. Vimentin radial density was calculated using the Radial Profile ImageJ plugin and normalized to radius equal to 1. Each analysis consisted of high resolution images at 400× total magnification of cross sections of different organoids.

The Roche In Situ Cell Death Detection Kit (Sigma) was used to visualize apoptotic cells in frozen sections of cardiac organoids based on the Roche protocol. Briefly, cardiac organoid frozen sections were fixed with 4% paraformaldehyde in PBS for 20 min at room temperature. Following washing in PBS for 30 minutes, samples were incubated in a permeabilization solution (0.1% Triton X-100 and 0.1% sodium citrate in PBS) for 2 minutes on ice. Then 50 μl of the TUNEL reaction mixture were added to samples and incubated at 37° C. for 1 hr. After washing in PBS (2 times at 5 min), nuclei were counterstained with DAPI (Molecular Probes/Invitrogen) diluted in PBS for 15 min at ambient temperature. Following the final wash procedure (PBS, 2 times at 5 min), glass cover slips were added to the slides using Fluoro-Gel (Electron Microscopy Sciences). TCS SP5 AOBS laser scanning confocal microscope (Leica Microsystems) was used for imaging.

NADH autoflourescence imaging of live cardiac organoids was performed in media at 37° C. within 1 hour of removal from culture conditions using an Olympus FV1200 laser scanning two-photon fluorescence microscope, which is equipped with a tunable ultrafast laser (Maitai, Newport) and two GaAsP PMTs. The excitation wavelength was tuned to 730 nm for autofluorescence imaging and a filter separated fluorescence with a passing band of violet (420-460 nm), which selected for NAD(P)H fluorescence.²² NADH index was calculated as the mean grey value of the sample (30-40 μm below surface) NADH autofluorescence minus the background mean grey value minus the mean grey value of the dead sample NADH autofluorescence.

### Example 9

### Mechanical Testing Using Micropipette Aspiration

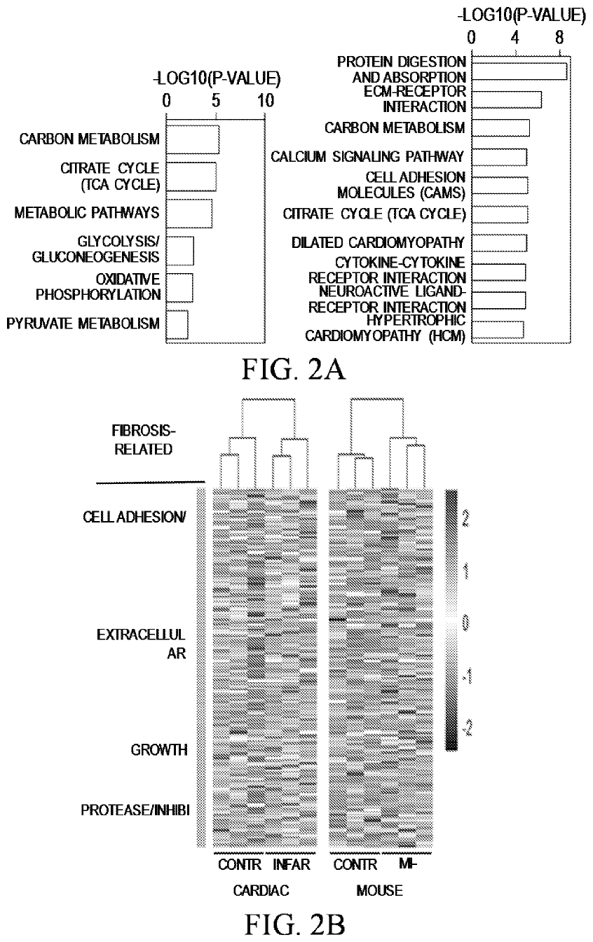

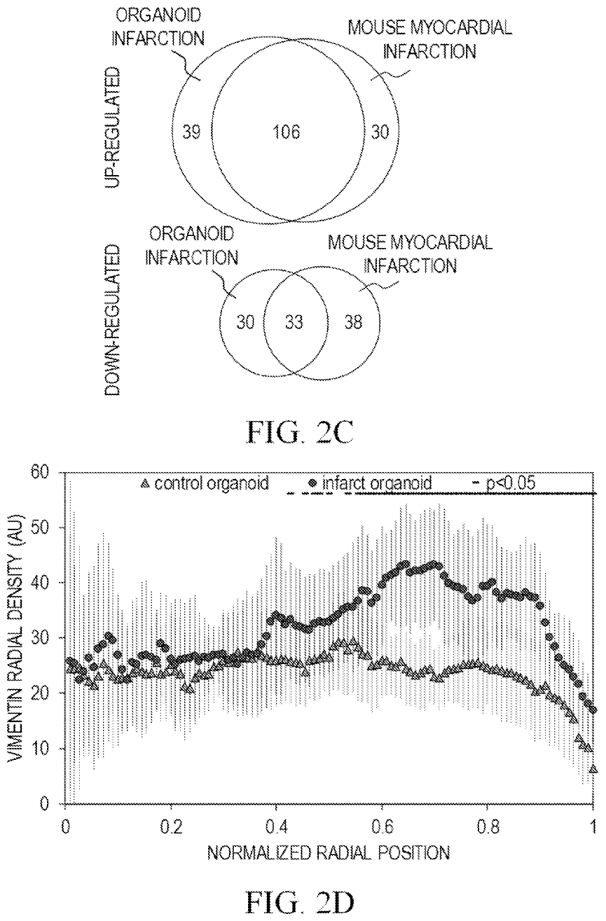

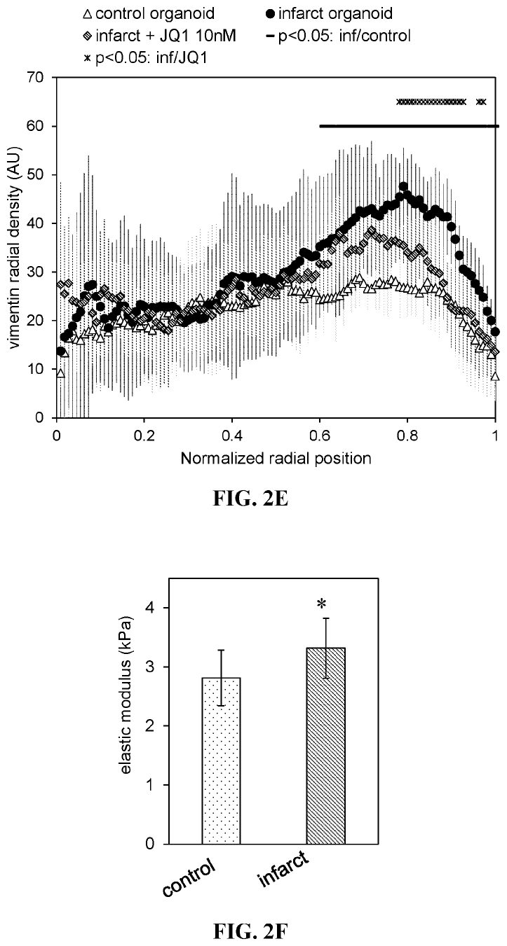

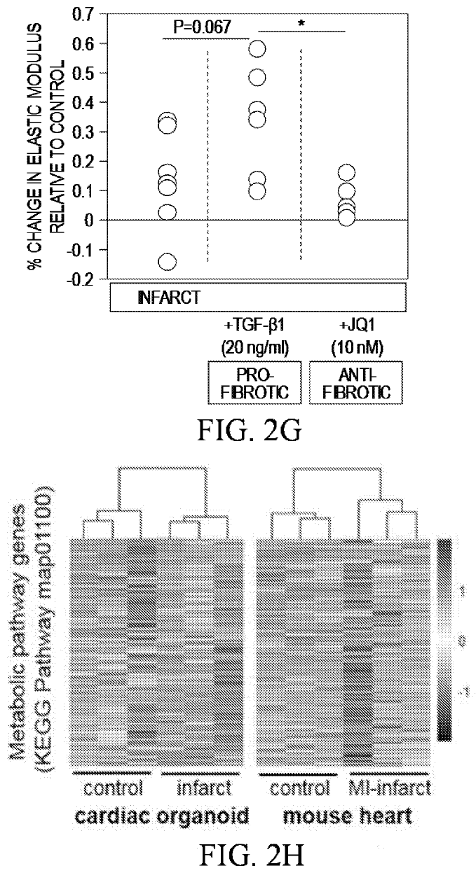

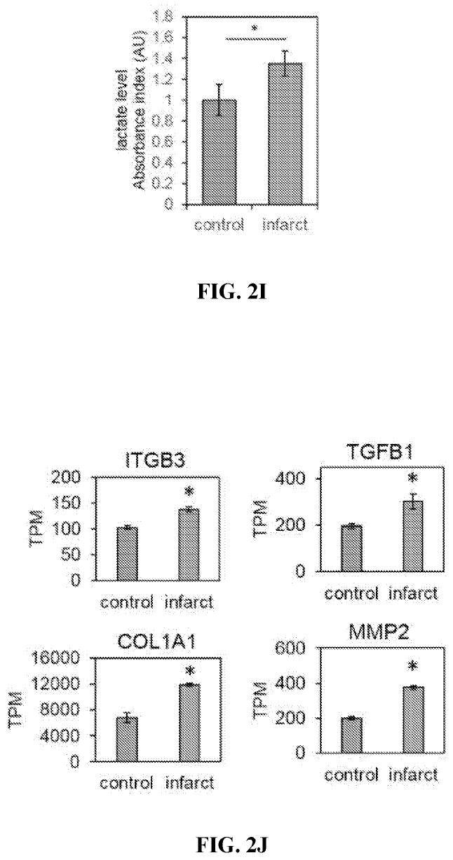

A micropipette aspiration was performed in media similarly to previous studies using a custom-built fluid reservoir to generate a fixed pressure of 40 cm H₂O (about 3.9 kPa) in pulled micropipette to apply the suction force on test organoids.^{23,24} Validation and stability of pressure changes were confirmed using an in-line 5 kPa 2-port pressure transducer with about 1 Pa sensitivity (Honeywell, Morristown, N.J.). Micropipettes were pulled to a final inner diameter of approximately 40-60 μm. Prior to and during testing, organoids were soaked in a 30 mM solution of 2,3-butanedione monoxime (BDM) (Sigma) in media for 5-10 min to eliminate contractions to reduce the effect of contractile status on tissue stiffness. Pressure was applied to the organoid surface and pictures were recorded until reaching equilibrium deformation (about 5 min) (FIG. 2F). Under the homogeneous half-space model assumptions²³, the change in length (L) from pre-deformation to equilibrium deformation was used to calculate the elastic modulus according the previously established relationship: E=3aΔp/2πL Φ(η), where E is elastic modulus, a is the inner micropipette radius, Δp is the applied pressure, Φ( ) is the wall function (under punch model assumptions), and η is the wall parameter.^{24,25}

### Example 10

### Discussion

Research surrounding the human myocardium after MI has been limited to chronic end-stage ischemic cardiomyopathy (given the availability of donor tissue) and in vitro human models of related cellular mechanisms (e.g., adrenergic stimulation, cell death from reperfusion) (Tiburcy et al., 2017 Circulation 135: 1832-1847; Ulmer et al., 2018 Stem Cell Reports 10: 834-847; Prat-Vidal et al., 2013 PloS One 8: e54785; Tarnayski et al., 2004 Physiol. Genomics 16: 349-360; Chen et al., 2018 Tissue engineering: Part A 25(9-10):71-724). Whereas previous cardiac 2D/3D systems have contributed to the understanding of effects of individual MI-related factors (e.g., hypoxia, adrenergic stimulation, substrate mechanics) in homogenous (i.e., no gradient) environments, this study leveraged nutrient transport principles (e.g., oxygen diffusion) in 3D microtissues alone with chronic adrenergic stimulation to create a gradient of “apoptotic center-dysfunctional interior-functional edge” in human cardiac organoids, which recapitulate the “infarct-border-remote zones” of infarct hearts. This allowed the organoids to mimic organotypic myocardial response to infarction.

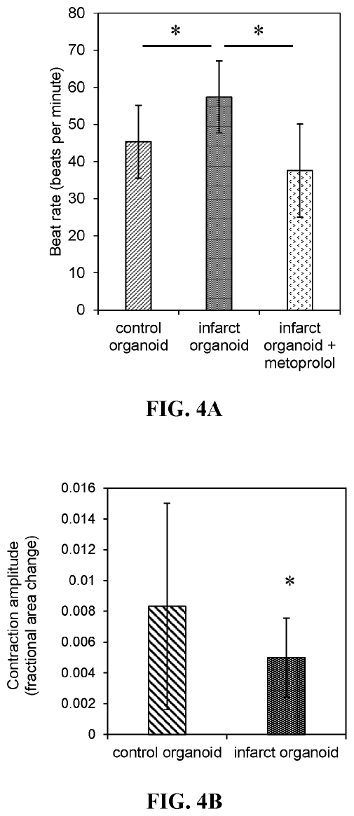

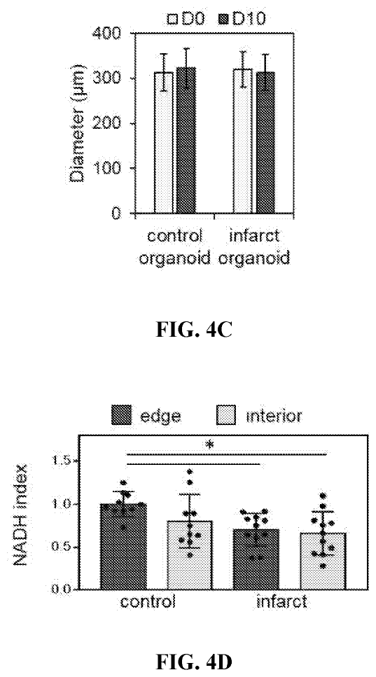

To gain an insight into oxygen distribution in cardiac organoids, a mathematical diffusion model was constructed using a 300 μm cardiac microtissue.¹⁴ In contrast to normoxia (20% oxygen), the microtissue in hypoxia (10% oxygen) experiences a gradient of viable to non-viable oxygen levels from edge to center (FIG. 1B-1C), mimicking gradual change in the nutrient availability in infarcted hearts.¹⁵ Applying this model, cardiac organoids cultured at 10% O₂ with 1 μM norepinephrine (NE) (i.e., infarct organoids) or in 10% O₂ only for 10 days showed apoptotic TUNEL+ staining in the center of organoid sections, attributed to the non-viable oxygen levels experienced at the center. Apoptotic TUNEL+ staining was carried out in control and infarct organoid using frozen sections (10% oxygen+1 μM norepinephrine) showing apoptotic core in infarct organoids. In situ imaging of live organoids also showed decreased NADH autofluorescence at the interior of the microtissues, supporting the hypoxia environment in the center of organoids (FIG. 4D).¹⁶ The infarct organoids also showed a NE-induced increase in beat rate, which was reversed when cultured with 10 μM metoprolol beta-adrenergic blocker, and a reduced contraction amplitude compared to controls (FIG. 4A-4B). Control and infarct organoid diameters were the same (FIG. 4C). 10 days of 0.1% O₂ with 1 μM NE culture of cardiac organoids resulted in microtissues with a vimentin+ fibrotic shell and apoptotic (TUNEL+) core, as well as the complete loss of hiPSC-CMs, showing a lack of αSA+ sarcomeric banding and no contractile function. This demonstrates the advantage of using a gradient-generating level of oxygen (e.g., 10%) instead of using a homogenous hypoxia treatment (e.g., 0.1% O₂) to create a functional tissue-level model of the post-infarct state.

To examine the global downstream effects of the cardiac infarction protocol (i.e., 10% O₂ and 1 μM NE treatment) on gene expression, the transcriptomes from the control and infarct organoids were analyzed using RNA sequencing (RNAseq). When compared to ischemic cardiac injury transcriptomic data from public human (i.e., human ischemic dilated cardiomyopathy, ICM) and animal (i.e., 1 wk MI) studies, infarct organoid differentially expressed (DE) genes overlapped with >1,000 genes of human, mouse, and porcine DE genes, similar to the overlap between animal MI models and human ICM samples (FIG. 1E).

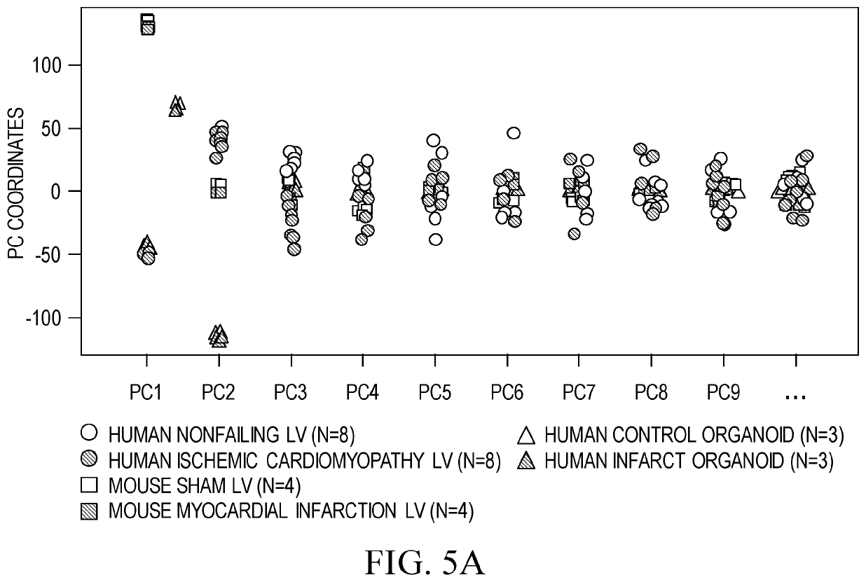

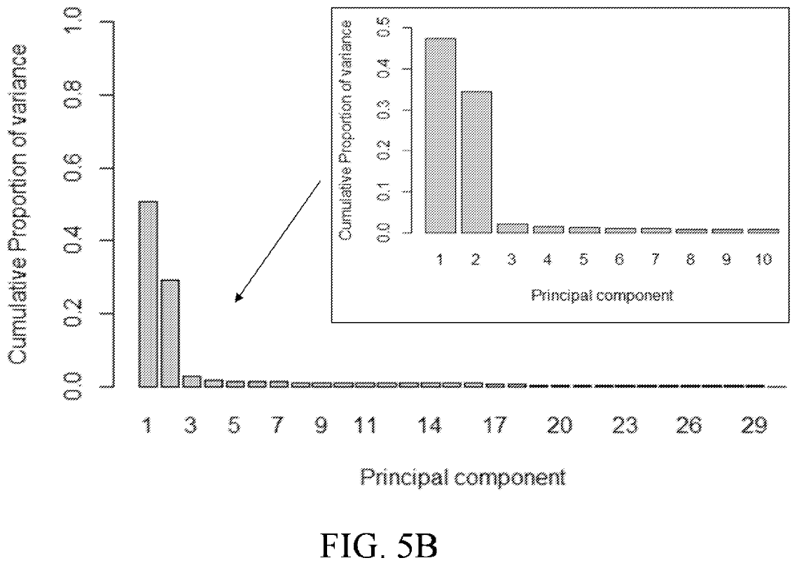

Whole transcriptome comparison between the organoid, human, and mouse data was performed using principal component analysis (PCA). After PCA of the 4,765 shared genes from the organoid data and two public RNA-seq datasets of human ICM and mouse MI (2 wks), the top PCs showed visible separations between samples (FIGS. 1F, 5A-5B). To functionally interpret these visual patterns with gene ontology, gene set enrichment analysis (GSEA) was used with the gene list and PC gene loadings as ranks (Table 6). PCI visualized the global transcriptomic variation between species, where organoid samples grouped with human samples separate from mouse (Left panel in FIG. 1F). Secondary analysis using a control mouse heart sample with different sequencing platform (to control for possible sequencing platform-based variance) confirmed species separation (FIG. 5B). This and the high proportion of variance (50.6%) of the “species” PC1 highlighted the translational insight of human cardiac organoids. PC2 separated heart tissue samples (positive) from cardiac organoid samples (negative), attributed to differences in tissue complexity, where immune system and developmental process terms were enriched in positive and negative PC2 gene loadings, respectively (Left panel of FIGS. 1F, 5C, Table 6). Plotting PC3 versus PC4 visualized a clear grouping of injury samples relative to controls across the x-axis (PC3), while PC4 showed separate grouping patterns of mouse and organoid control/injury samples in contrast to a lack of separation of human control and ICM samples across the y-axis (PC4) (Right panel of FIG. 1F). Gene ontology of loadings-ranked PC3 genes supported the ischemic cardiac injury phenotype (e.g., extracellular matrix, leukocyte migration, TGF-beta receptor binding) of injury samples (negative) and physiological phenotype (e.g., cellular respiration, regulation of conduction) of control samples (positive) (Table 1, Table 6). PC4 characterization revealed that mouse and organoid injury samples shared positive PC4 coordinate locations with associated functional terms indicative of acute infarct injury (e.g., regulation of inflammatory response, cell chemotaxis), while the human injury samples had dispersed coordinates along PC4 (Table 2, Table 6), attributed to their large biological variation (e.g., time after injury, disease severity, tissue isolation method, age). By incorporating new dimensions (i.e., human and mouse heart failure data), PCA delineated the dimensions where relevant pathological similarities exist (PC3, PC4), yet acknowledged inherent differences between species and tissue complexity (PC1, PC2). Overall, meta-analysis of the infarct organoid transcriptome with ischemic heart failure data from multiple species established a systems-level relevance of the cardiac infarct organoids in modeling human myocardial infarction.

With a translational relevance, we next investigated characteristics of ischemic cardiac injury observed in the organoid infarction platform. Fibrosis is one of the central hallmarks of ischemic heart failure in humans and animal MI models.¹⁷ Pathway analysis of DE genes in infarct organoids showed top hits that included “carbon metabolism” (p=5.24×10⁶), “citrate cycle (TCA)” (p=9.25×10⁻⁶), and “glycolysis/gluconeogenesis” (p−1.67×10⁻³) (FIG. 2A). Notably, transcriptomic shifts of DE genes in “metabolic pathways” (KEGG pathway map01100), a large pathway term including several metabolic modules, in infarct organoids were consistent with data from mouse 1 wk post-MI samples (FIG. 2H). These changes supported a biomimetic shift towards anaerobic metabolism due to the organoid infarction protocol. This was further supported by significantly increased L-lactate levels, an accumulated metabolic-by-product, in infarct organoid media compared to control organoid media (FIG. 2I). Further comparison of infarct to control organoid gene ontology of DE genes indicated additional top significant biological processes related to multicellular interactions and extracellular matrix (ECM) and top pathway hits of “ECM-receptor interaction” and “dilated cardiomyopathy” (FIG. 2A, Tables 3 and 4). An assembled fibrosis-related gene set, containing genes related to cell adhesion/migration (e.g., ITGB3), ECM (e.g., COL1A1), growth factors (e.g., TGFB1), and protease/inhibitor (e.g., MMP2) (Table 7; FIG. 2J), showed an overall increase in fibrosis-related gene expression in infarct organoid samples, consistent with data from mouse 1 wk post-MI samples (FIGS. 2B-2C). Without being bound to theory, while fibrotic gene shifts may have partially resulted from the increased fibroblast to cardiomyocyte ratio due to death of cardiomyocytes, these changes are typical for the damaged regions of the heart in vivo as well and thus support tissue-level transcriptomic comparisons (e.g., between organoids and mouse heart tissue).

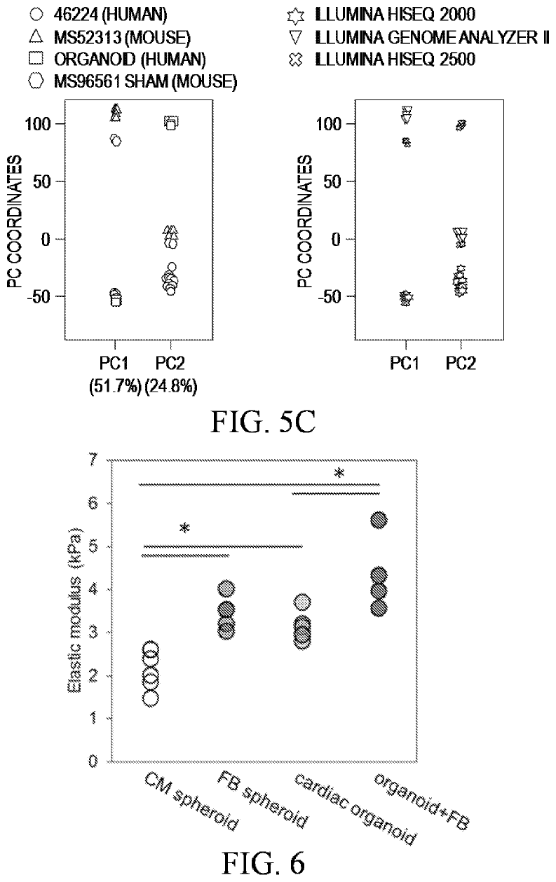

The transcriptomic changes indicated a biomimetic fibrosis-like downstream response within the infarct organoids. This data was further characterized by structural analysis of fibroblast cellular organization. Infarct organoids showed a significant shift in vimentin+ organization (i.e., fibroblasts) toward the edge of the organoid compared to control organoids, seen by confocal imaging and radial density plots of vimentin+ area in organoid frozen sections (FIG. 2D). The presence of myofibroblasts is commonly used to histologically identify fibrotic tissue in the infarcted heart.¹⁷ Infarct organoids showed numerous myofibroblast-like structures, marked by elongated, phalloidin+/alpha smooth muscle actin+ (αSMA) phenotype in contrast to control organoids using immunofluorescence imaging techniques. The presence of myofibroblast-like cells and associated fibrotic gene profile suggested a tissue-level change in cardiac organoid mechanical environment. A micropipette aspiration method was adapted for microtissues to measure the elastic modulus (i.e., stiffness) of the outer viable regions of the infarct organoids.¹⁸ The stiffness was significantly increased in infarct organoids over control organoids, similar to mechanical changes seen in infarcted myocardial tissue (FIG. 2E).¹⁹ In vivo studies have shown that mechanical changes in injured fibrotic hearts in acute MI reflect the total effect of fibroblast-associated changes (e.g., cell density, total ECM deposition/remodeling).^{19,20} Supporting this, our mechanical testing of newly formed microtissue variants showed that cardiomyocyte (CM) spheroids had the lowest stiffness and was increased in fibroblast (FB) spheroids, cardiac organoids, and cardiac organoids with 10% more FBs (organoid+FB) (FIG. 6).

Incorporation of TGF-β1 into the infarction protocol as a pro-fibrotic stimulus further increased collagen organization and the presence of αSMA+ myofibroblast-like cells with an associated significant increase in organoid stiffness. Immunoflouresent staining of myofibroblast-like cells in organoid sections using a pro-fibrotic stimulus of TGF-bl (20 ng/mL) during infarction protocol showed a visible shift in aSMA/F-actin (phalloidin) colocalization with fibrillary structure. (FIG. 2F). Furthermore, application of a recent anti-fibrotic epigenetic drug for heart failure, JQ 1 bromodomain inhibitor, to infarct organoid culture resulted in a significant decrease in stiffness and significantly reduced the vimentin+ density at the infarct organoid edge (FIGS. 2F-2G).²¹ The change in the tissue-level mechanical properties alongside fibrotic transcriptomic shifts and presence of αSMA+ myofibroblast-like cells corroborate an endogenous biomimetic fibrosis response in infarct organoids. While previous studies have required fibroblast monoculture, the addition of TGF-β1, and/or direct changes to culture material properties to investigate fibrotic/myofibroblast behavior²²⁻²⁴, this is the first observation of endogenous increases in tissue stiffness and corresponding presence of myofibroblast-like cells in cardiac organoids as a result of an upstream pathological stimuli, supporting the relevance of the organoid model of cardiac infarction.

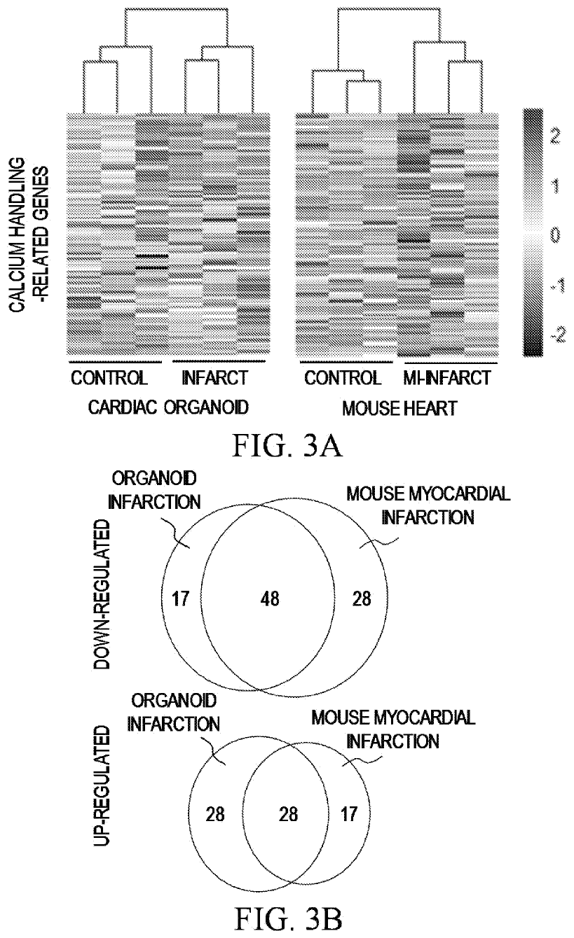

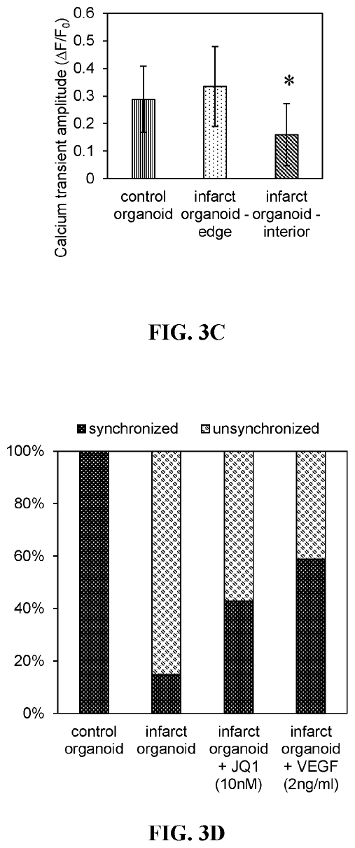

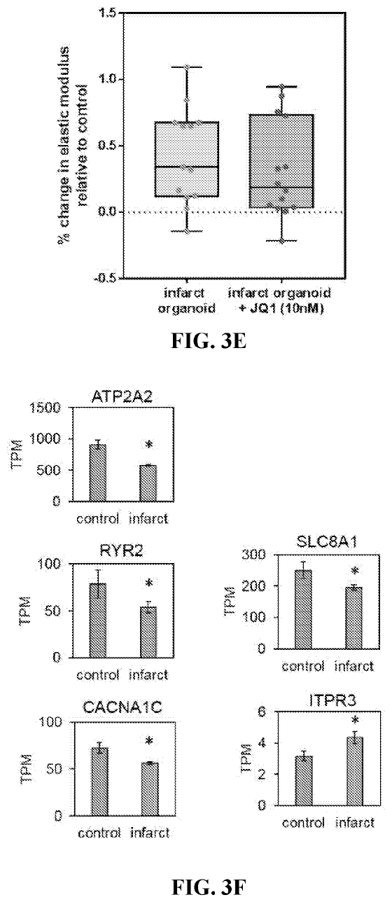

In addition to fibrosis, calcium handling changes associated with MI serve as a functional hallmark of ischemic cardiac injury.²⁵ Pathological calcium handling is linked to contractile dysfunction and contributes to the occurrence of arrhythmias after MI.²⁶ At the transcriptomic level, “ion transport” was a top ten gene ontology term between control and infarct organoids, and top pathway hits included “calcium signaling pathway” and “arrhythmogenic right ventricular cardiomyopathy”, indicative of changes in the calcium handling (Tables 3-5). The “calcium signaling pathway” KEGG term 4020 was used to assemble a calcium handling-related gene set (Table 8). After filtering for the calcium handling-related gene set, infarct organoids showed an overall consistency with calcium handling gene expression of 1 wk post-MI mouse heart samples, including significant decreases in well-studied calcium-handling components ATP2A2 (sarco-endoplasmic reticulum Ca²⁺-ATPase), RYR2 (ryanodine receptor), CACNA1C (L-type calcium channel), SLC8A1 (sodium-calcium exchanger) and increase in ITPR3 (inositol 1,4,5-triphosphate receptor type 3) (FIGS. 3A, 3B, and 3F). These transcriptomic changes suggested a biomimetic pathological response in calcium handling within the infarct organoids.

To functionally interpret the transcriptomic shifts in cardiomyocyte calcium handling and arrhythmogenesis, a two-photon, laser-scanning, light sheet (2PLS) microscope was used that allowed for deeper tissue penetration with high-speed imaging (50 frames/sec) and orthogonal selected plane (about 4 μm thickness) illumination.²⁷ Given its strength for in situ imaging, the 2PLS microscope allowed for the visualization of calcium handling in the interior regions of 3D cardiac microtissues to study calcium handling and arrhythmogenicity across the organoids. Organoids were fabricated with GCaMP6-labeled hiPSC-CMs and calcium transient amplitudes (ΔF/F₀) were measured from “cell-sized” regions of interest (ROIs) (representing individual cardiomyocytes) inside organoids. Imaging of control organoids displayed synchronized beating with an interconnected cardiomyocyte network. Specifically, imaging and calcium transient profiles of control and infarct organoid from selected imaging planes at >50 μM below organoid surface were carried out which showed unsynchronization of edge and interior cardiomyocytes regions of interest in infarct organoids. In contrast, imaging of infarct organoids revealed notable unsynchronized beating profiles (i.e., arrhythmias) between separated cardiomyocyte populations at the interior and the edge of the infarct organoids. Interior cardiomyocytes in the infarct organoids showed significantly lower max calcium transient amplitude in contrast to the control and infarct edge cardiomyocytes. In particular, imaging and calcium transient profiles of synchronized edge and interior cardiomyocyte ROIs from human induced pluripotent stem cell-derived-cardiomyocyte (hiPSC-CM) spheroids were cultured in the cardiac organoid infarction protocol. The difference in calcium synchronization between control and infarct organoids was supported by segregated αSA+ cardiomyocyte populations in infarct organoids in contrast to interconnected αSA+ cardiomyocytes in the control organoids was demonstrated using immunofluorescent staining of organoid sections showing interconnected alpha sarcomeric actinin (αSA)-positive cardiomyocytes in control organoids and separation of edge and interior cardiomyocytes by vimentin-positive cells in infarct organoids. We reasoned that fibrosis (i.e., vimentin+ cell-associated changes) separated interior cardiomyocytes in infarct organoids into an unsynchronized, smaller beating population that may experience hypoxia-induced aberrations in calcium handling, consistent with the in vivo contributors to ventricular arrhythmia post-MI.²⁶ This was supported by hiPSC-CM-only spheroids (i.e., without fibroblasts) cultured in the infarction protocol that showed no indication of unsynchronized beating nor differences in edge-interior calcium transient amplitudes (FIG. 7). In addition, anti-fibrotic drug (JQ1) and pro-angiogenic drug (VEGF) treatment of infarct organoids resulted in a reduction of unsynchronization and a beating cardiomyocyte population with improved synchronization (FIGS. 3A-3E).

Previous research has shown that hiPSC-CMs from breast cancer patients with chemotherapy-induced cardiotoxicity were more sensitive to doxorubicin (DOX), a known cardiotoxic anticancer medication, than breast cancer patients without chemotherapy-induced cardiotoxicity, suggesting genetic basis for DOX-based cardiotoxicity (Benjamin et al., 2017 Circulation 135: 1832-1847). As a control, we performed 2D studies using hiPSC-CMs with DOX to evaluate the combined effects of an in vitro infarction protocol and DOX on hiPSC-CMs. Hypoxic culture (1%) with 1 μM NE in organoid media for 2 days prior to the 2 days DOX treatment causes an exacerbation of DOX-induced reduction in viability and reduction in contractile structures/organization. Nearly 100% of cells death was found for hiPSC-CMs after 4 days of the same hypoxic culture using hiPSC-CM media with galactose but no glucose, which has been attributed to the lack of glucose in the hiPSC-CM media. In addition, extended culture of 2D hiPSC-CMs in organoid media to mimic prolonged organoid infarction protocol led to minimal and irregular hiPSC-CM contractile behavior accompanied with the reduction of αSA+ cells. In contrast to the inherent difficulties of using 2D hiPSC-CM culture systems to model post-MI responses, human infarct organoids provide a robust, biomimetic 2D tissue-level context to evaluate the effects of DOX on the post-MI cardiac tissues.

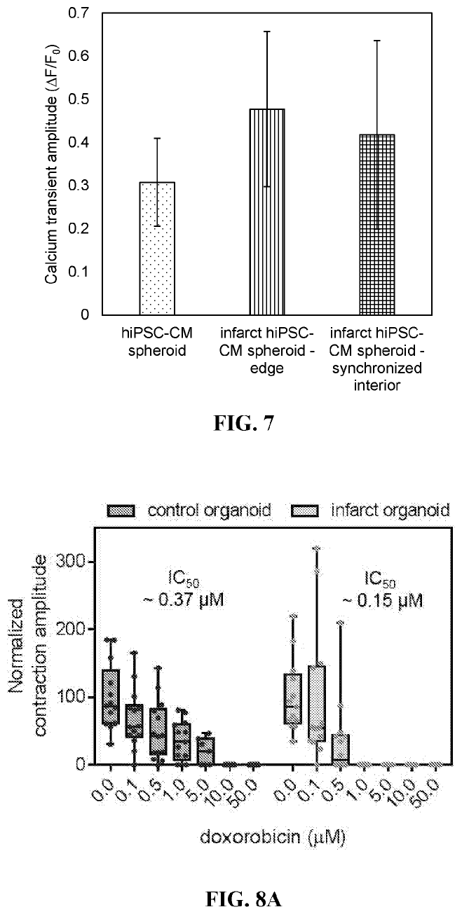

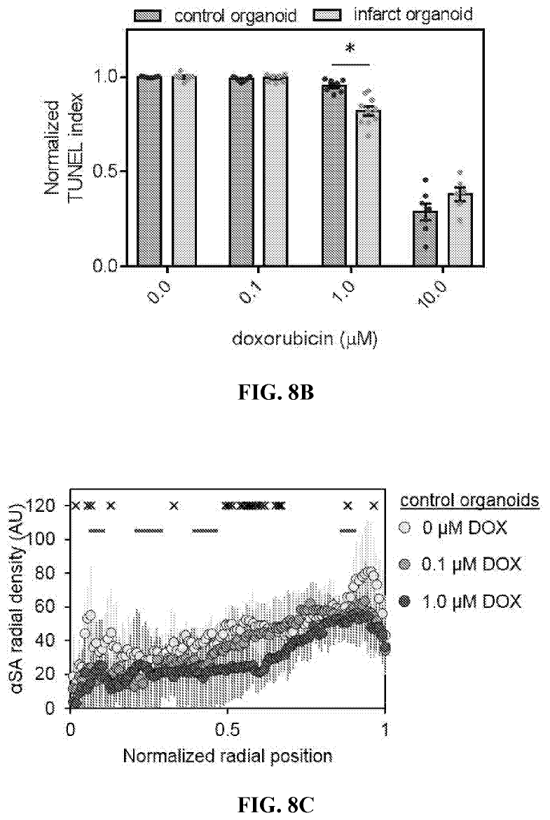

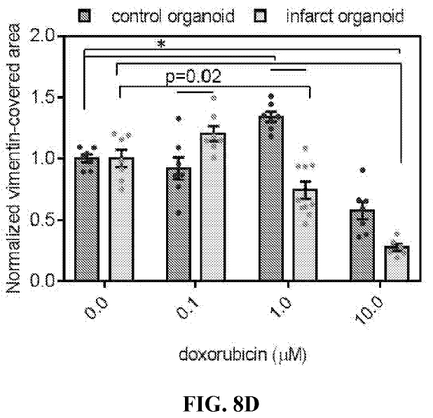

Application of DOX showed functional toxicity with a reduced IC₅₀ of contraction amplitude in infarct organoids (˜0.15 μM) compared to control organoids (˜0.37 μM) (FIG. 8A). This was further supported by cessation of beating in infarct and control organoids at 1 μM and 10 μM, respectively. The observed detrimental effects of DOX on contractile function in both control and infarct organoids is consistent with the clinical data showing decreased contractile function (i.e., left ventricular ejection fraction) after anthracycline exposure (Narayan, et al., 2017 Circulation 135: 1397-1412). The reduction in organoid contraction was supported by the TUNEL analysis that showed significantly increased apoptosis for infarct organoids at 1 μM (FIG. 8B). Furthermore, infarct organoids displayed a more severe disarray of sarcomeric structures across D space (i.e., exterior to interior) compared to control organoids with increasing dose of DOX, where αSA staining significantly decreases relative to vehicle control more notably at the interior of infarct organoids at 0.1 μM in contrast to control organoids (FIG. 8C), consistent with the cdecreased contraction amplitude at low doses in infarct organoids. In addition to hiPSC-CM specific changes, DOX exposure induced an increase in vimentin+ density at a lower dose in infarct organoids (0.1 μM) than in control organoids (1.0 μM) (FIG. 8D), indicating a similar phenotype to DOX-induced cardiac fibrosis. This is supported by histopathological evidence of fibrosis in myocardial biopsies from children and adults with anthracycline cardiotoxicity. Overall, the use of cardiac infarct organoids in cardiotoxicity screening demonstrated that pre-existing (hypoxic) cardiac injury exacerbates the cardiotoxicity of DOX, in line with worsening heart failure of anthracycline-treated cancer patients with pre-existing cardiovascular risk. While not wishing to be bound to theory, the worsened phenotype of infarct organoids in response to DOX was reasoned to be attributed to pre-existing oxidative stress in infarct organoids evidenced by the previously mentioned metabolic shifts, hypoxia staining, and increased DNA damage as marked by TUNEL staining in the middle of infarct organoids in combination with the reported differential effect of DOX on different metabolic/oxidative states (Burridge et al., 2016 Nat. Med. 22: 547-556). These results demonstrated that human cardiac organoids, for the first time, allowed for the recapitulation of 3D tissue-level responses, including cardiac and fibrotic effects, to drug-induced/exacerbated cardiotoxicity.

By leveraging nutrient diffusion limitations in 3D microtissues, we developed the first human cardiac organoid disease model that recapitulates the major hallmarks of myocardial infarction at a transcriptomic, structural and functional level. While human organoids have been widely used to study diseases caused by genetic mutation, this is the first demonstration of the use of tissue engineering principles (i.e., nutrient transport) to design an in vitro organotypic disease model with non-genetic upstream pathological stimuli. It is our belief that the focus on upstream pathological stimuli allowed for the recapitulation of major hallmarks of human myocardial infarction.

Through the integration of robust infarct organoid fabrication and imaging-based function analysis, we established a platform amenable for heart failure drug screening. The transcriptome- to function-level changes provided a multi-dimensional validation that illustrates the extent to which infarct organoids recreate responses of human cardiac tissue after infarction. As human cardiac tissues post-MI are difficult to obtain²⁸, human cardiac infarct organoids offer personalized models for precision cardiovascular medicine and future investigation into genetic contributions of patients' tissue-level response to myocardial infarction. While organoids have traditionally been prepared with embryoid bodies, this study demonstrated that the self-assembly of tissue-specific cell types provides a powerful alternative to prepare organoids with tissue-mimetic transcriptome, structure and function.²⁹

The foregoing is illustrative of the present invention, and is not to be construed as limiting thereof. The invention is defined by the following claims, with equivalents of the claims to be included therein.

TABLE 1
PC3 gene ontology

| | Normatized | NOM |
|---|---|---|
| | enrichment score | p-value |
| Positive (10) | | |
| mitochondrial protein complex | 1.88 | 0.00 |
| cellular respiration | 1.83 | 0.00 |
| regulation of cardiac conduction | 1.83 | 0.00 |
| Negative (430) | | |
| extracellular matrix | −2.00 | 0.00 |
| cell chemotaxis | −2.04 | 0.00 |
| cytokine activity | −1.03 | 0.00 |
| angiogenesis | −1.91 | 0.00 |
| neurotransmitter transport | −1.86 | 0.00 |
| integrin binding | −1.88 | 0.00 |
| leakocyte migration | −1.05 | 0.00 |
| TGF-beta recepter binding | −1.85 | 0.00 |
| regulation of extrinsic apoptotic | 4.04 | 0.00 |
| signaling pathway | | |
| developmental cell growth | −1.83 | 0.00 |

TABLE 2
PC4 gene ontology

| | Normalized | NOM |
|---|---|---|
| | enrichment score | p-value |
| Positive (443) | | |
| myeloid leukocyte migration | 2.23 | 0.00 |
| immune response | 2.20 | 0.00 |
| regulation of inflammatory response | 2.14 | 0.00 |
| leukocyte activation | 2.14 | 0.00 |
| cell chemotakis | 2.13 | 0.00 |
| lymphocyte activation | 2.12 | 0.00 |
| regulation of immune response | 2.10 | 0.00 |
| regulation of response to wounding | 2.09 | 0.00 |
| innate immune response | 2.06 | 0.00 |
| extracellular matrix | 2.05 | 0.00 |
| Negative (33) | | |
| oxidative phosphorylation | −1.80 | 0.00 |
| regulation of neurotransmitter secretion | −1.77 | 0.00 |
| cellular respiration | −1.69 | 0.00 |

TABLE 3

| Pathway | p-value |
|---|---|
| Protein digestion and absorption | 2.74E−09 |
| ECM-receptor interaction | 5.32E−07 |
| Carbon metabolism | 5.24E−56 |
| Calcium signaling pathway | 8.85E−06 |
| Cell adhesion molecules (CAMs) | 9.10E−06 |
| Citrate cycle (TCA cycle) | 9.25E−06 |
| Dilated cardiomyopathy | 9.81E−06 |
| Cytokine-cytokine receptor interaction | 1.11E−05 |
| Neuoroactive ligand-receptor interaction | 1.16E−05 |
| Hypertrophic cardiomyopathy (HCM) | 1.91E−05 |

TABLE 4

| | # of DE | |
|---|---|---|
| Gene ontology term | genes | p-value |
| multicellular organismal process | 1759 | 1.00E−24 |
| single-organism process | 3202 | 1.00E−24 |
| single-multicellular organism process | 1623 | 6.20E−24 |
| extracellular matrix organization | 155 | 9.60E−21 |
| extracellular structure organization | 158 | 1.60E−20 |
| system process | 478 | 4.20E−19 |
| single-organism cellular process | 2914 | 1.00E−18 |
| ion transport | 439 | 2.10E−17 |
| cell differentiation | 1013 | 2.10E−17 |
| multicellular organism development | 1346 | 2.50E−17 |

TABLE 5

| Pathway | p-value |
|---|---|
| Protein digestion and absorption | 2.74E−09 |
| ECM-receptor interaction | 5.32E−07 |
| Carbon metabolism | 5.24E−06 |
| Calcium signaling pathway | 8.85E−06 |
| Cell adhesion molecules (CAMs) | 9.10E−06 |
| Citrate cycle (TCA cycle) | 9.25E−06 |
| Dilated cardiornyopathy | 9.81E−06 |
| Cytokine-cytokine receptor interaction | 1.11E−05 |
| Neuroactive ligand-receptor interaction | 1.16E−05 |
| Hypertrophic cardiomyopathy (HCM) | 1.91E−05 |
| Metabolic pathways | 2.46E−05 |
| Malaria | 2.87E−05 |
| Alzheimer's disease | 3.66E−05 |
| Arrhythmogenic right ventricular cardiomyopathy (ARVC) | 4.18E−05 |
| Parkinson's disease | 6.26E−05 |
| Nicotinate and nicotinamide metabolism | 1 19E−04 |
| Glutamatergic synapse | 1.25E−04 |
| Cardiac muscle contraction | 1.86E−04 |
| Proteoglycans in cancer | 1.88E−04 |
| African trypanosomiasis | 7.21E−04 |

TABLE 6

| | | | | |
|---|---|---|---|---|
| | | Normalized | | |
| Gene ontocology (GO) term | # of genes | enrichment score | NOM p-value | FDR q-value |

Principal Component 1

| | | | | |
|---|---|---|---|---|
| Positive (1) | | | | |
| positive regulation of peptidyl tyrosine phosphorylation | 49 | 1.94 | 0.00 | 0.16 |
| Negative (0) | | | | |

Principal Component 2

| | | | | |
|---|---|---|---|---|
| Positive (174) | | | | |
| cytokine production | 37 | 2.18 | 0.00 | 0.00 |
| activation of immune response | 108 | 2.05 | 0.00 | 0.01 |
| immune response regulating cell surface receptor signaling pathway | 77 | 1.97 | 0.00 | 0.04 |
| antigen recptor mediated signaling pathway | 51 | 1.96 | 0.00 | 0.04 |
| i kappab kinase nf kappab signaling | 23 | 1.91 | 0.00 | 0.07 |
| innate immune response activating cell surface receptor signaling pathway | 30 | 1.89 | 0.00 | 0.07 |
| activation of innate immune response | 61 | 1.89 | 0.00 | 0.06 |
| cytosolic ribosome | 25 | 1.88 | 0.00 | 0.07 |
| organic acid catabolic process | 65 | 1.88 | 0.00 | 0.07 |
| myeloid leukocyte activation | 34 | 1.87 | 0.00 | 0.06 |
| carbon carbon lyase activity | 17 | 1.87 | 0.00 | 0.06 |
| positive regulation of immune response | 146 | 1.87 | 0.00 | 0.06 |
| glutathione transferase activity | 17 | 1.87 | 0.00 | 0.05 |
| alpha amino acid catabolic process | 31 | 1.83 | 0.00 | 0.08 |
| cellular amino acid catabolic process | 39 | 1.83 | 0.00 | 0.08 |
| intramolecular oxidoeductase activity | 17 | 1.82 | 0.00 | 0.08 |
| leukocyte cell cell adhesion | 75 | 1.82 | 0.00 | 0.08 |
| structural constituent of ribosome | 64 | 1.82 | 0.00 | 0.06 |
| signaling adaptor activity | 21 | 1.81 | 0.00 | 0.08 |
| immune response | 253 | 1.81 | 0.00 | 0.06 |
| electron transport chain | 44 | 1.80 | 0.00 | 0.09 |
| inner mitochondrial membrane protein complex | 49 | 1.79 | 0.00 | 0.08 |
| defense response to bacterium | 44 | 1.79 | 0.00 | 0.09 |
| phagocytic vesicle | 23 | 1.77 | 0.00 | 0.10 |
| organic cyclic compound catabolic process | 116 | 1.77 | 0.00 | 0.10 |
| cellular respiration | 59 | 1.77 | 0.00 | 0.10 |
| ribosomal subunit | 42 | 1.77 | 0.00 | 0.10 |
| organnitrogen compound catabolic process | 104 | 1.76 | 0.00 | 0.10 |
| adaptive immune response | 52 | 1.76 | 0.00 | 0.10 |
| organelle inner membrane | 186 | 1.75 | 0.00 | 0.10 |
| respiratory chain | 41 | 1.75 | 0.00 | 0.10 |
| lymphocyte activation | 95 | 1.75 | 0.00 | 0.10 |
| antigen processing and presentation of exogenous peptide | 19 | 1.74 | 0.01 | 0.10 |
| oxidoreductase activity acting on nadph quinone or similar compound | 23 | 1.74 | 0.00 | 0.11 |
| as acceptor | | | | |
| mitochondrial protein complex | 62 | 1.73 | 0.00 | 0.11 |
| Negative (114) | | | | |
| skin development | 54 | −2.03 | 0.00 | 0.03 |
| mitotic nuclear division | 90 | −1.97 | 0.00 | 0.04 |
| cell division | 127 | −1.94 | 0.00 | 0.05 |
| anterior posterior pattern specification | 62 | −1.92 | 0.00 | 0.06 |
| embryonic skeletal system development | 41 | −1.69 | 0.00 | 0.07 |
| metallopeptidase activity | 62 | −1.87 | 0.00 | 0.09 |
| regionalization | 92 | −1.86 | 0.00 | 0.09 |
| epithelial to mesenchymal transition | 18 | −1.84 | 0.00 | 0.11 |
| regulation of nuclear division | 45 | −1.84 | 0.00 | 0.10 |
| hormone activity | 33 | −1.83 | 0.00 | 0.10 |
| ossification | 74 | −1.83 | 0.00 | 0.09 |
| cell cell signaling | 222 | −1.83 | 0.00 | 0.08 |
| osteoblast differentiation | 42 | −1.82 | 0.00 | 0.09 |
| skeletal system development | 135 | −1.82 | 0.00 | 0.08 |
| neuropeptide hormone activity | 15 | −1.80 | 0.00 | 0.10 |
| metalloendopeptidase activity | 37 | −1.80 | 0.00 | 0.10 |
| reproductive system development | 129 | −1.79 | 0.00 | 0.10 |
| cell cycle g1 s phase transition | 40 | −1.78 | 0.00 | 0.12 |
| synaptic signaling | 128 | −1.77 | 0.00 | 0.11 |
| sensory perception of pain | 29 | −1.77 | 0.01 | 0.12 |
| regulation of synaptic plasticity | 44 | −1.76 | 0.00 | 0.12 |
| calcium channel complex | 16 | −1.76 | 0.00 | 0.12 |
| protein heterooligomerization | 26 | −1.75 | 0.00 | 0.12 |
| intermediate filament | 25 | −1.75 | 0.00 | 0.11 |
| signal release | 45 | −1.75 | 0.00 | 0.11 |
| skeletal system morphogenesis | 65 | −1.75 | 0.00 | 0.11 |
| mammary gland development | 34 | −1.73 | 0.00 | 0.13 |
| positive regulation of nuclear division | 15 | −1.73 | 0.01 | 0.13 |
| divalent inorganic cation transmembrane transporter activity | 64 | −1.73 | 0.00 | 0.13 |
| calcium ion tranmembrane transport | 54 | −1.72 | 0.00 | 0.13 |
| dna packaging | 38 | −1.72 | 0.00 | 0.13 |
| cell cycle phase transition | 79 | −1.72 | 0.00 | 0.13 |
| positive regulation of cell cycle arrest | 31 | −1.72 | 0.01 | 0.13 |
| regulation of cell cycle arrest | 37 | −1.71 | 0.00 | 0.14 |
| neuronal postsynaptic density | 20 | −1.71 | 0.00 | 0.14 |

Principal Component 3

| | | | | |
|---|---|---|---|---|
| Positive (10) | | | | |
| mitochondrial protein complex | 62 | 1.88 | 0.00 | 0.17 |
| mitochondrial transition | 43 | 1.86 | 0.00 | 0.15 |
| inner mitochondrial membrane protein complex | 49 | 1.86 | 0.00 | 0.12 |
| translational termination | 36 | 1.84 | 0.00 | 0.12 |
| cellular respiration | 59 | 1.83 | 0.00 | 0.11 |
| regulation of cardiac conduction | 23 | 1.83 | 0.00 | 0.09 |
| mitochondrial respiratory chain complex i biogenesis | 25 | 1.81 | 0.00 | 0.11 |
| oxidative phosphorylation | 40 | 1.79 | 0.00 | 0.13 |
| respiratory chain | 41 | 1.77 | 0.00 | 0.16 |
| hair cell differentiation | 15 | 1.74 | 0.01 | 0.19 |
| Negative (430) | | | | |
| extracellular matrix | 125 | −2.09 | 0.00 | 0.00 |
| osteoblast differentiation | 42 | −2.06 | 0.00 | 0.00 |
| taxis | 120 | −2.04 | 0.00 | 0.00 |
| cell chemotaxis | 37 | −2.04 | 0.00 | 0.00 |
| proteinaceous extracellular matrix | 107 | −2.00 | 0.00 | 0.01 |
| extracellular structure organization | 78 | −2.00 | 0.00 | 0.01 |
| glycoaminoglycan binding | 54 | −1.96 | 0.00 | 0.01 |
| fat cell differentiation | 38 | −1.95 | 0.00 | 0.01 |
| cytokine activity | 57 | −1.93 | 0.00 | 0.02 |
| regulation of biomineral tissue development | 19 | −1.92 | 0.00 | 0.02 |
| angiogenesis | 89 | −1.91 | 0.00 | 0.02 |
| extracellular matrix component | 39 | −1.91 | 0.00 | 0.02 |
| pressynaptic process involved in synaptic transmission | 29 | −1.90 | 0.00 | 0.02 |
| leukocyte chemotaxis | 28 | −1.89 | 0.00 | 0.02 |
| skeletal muscle cell differentiation | 20 | −1.89 | 0.00 | 0.02 |
| regulation of leukocyte migration | 48 | −1.88 | 0.00 | 0.03 |
| vasculature development aging | 152 | −1.87 | 0.00 | 0.03 |
| neurotransmitter transport | 44 | −1.86 | 0.00 | 0.03 |
| integrin binding | 29 | −1.86 | 0.00 | 0.03 |
| smad protein signal transduction | 17 | −1.86 | 0.00 | 0.03 |
| neuron projection development | 146 | −1.85 | 0.00 | 0.03 |
| regulation of neurotransmitter levels | 54 | −1.85 | 0.00 | 0.03 |
| transforming growth factor beta receptor binding | 15 | −1.85 | 0.00 | 0.03 |
| heparin binding | 41 | −1.85 | 0.00 | 0.03 |
| leukocyte migration | 75 | −1.85 | 0.00 | 0.03 |
| transmembrane receptor protein srine threonine kinase signaling pathway | 69 | −1.84 | 0.00 | 0.03 |
| aminoglycan catabolic process | 22 | −1.84 | 0.00 | 0.03 |
| response to molecule of bactterial origin | 108 | −1.84 | 0.00 | 0.03 |
| regulation of extrinsic apoptotic signaling pathway | 48 | −1.84 | 0.00 | 0.03 |
| developmental cell growth | 15 | −1.83 | 0.00 | 0.03 |
| response to external stimulus | 490 | −1.83 | 0.00 | 0.03 |
| myeloid leukocyte migration | 18 | −1.83 | 0.00 | 0.03 |
| signal release | 45 | −1.82 | 0.00 | 0.03 |
| branching morphogenesis of an epithelial tube | 42 | −1.82 | 0.00 | 0.03 |

Principal Component 4

| | | | | |
|---|---|---|---|---|
| Positive (443) | | | | |
| myeloid leukocyte migration | 18 | 2.23 | 0.00 | 0.00 |
| leukocyte chemotaxis | 28 | 2.20 | 0.00 | 0.00 |
| immune response | 253 | 2.20 | 0.00 | 0.00 |
| leukocyte migration | 75 | 2.18 | 0.00 | 0.00 |
| regulation of inflammatory response | 81 | 2.14 | 0.00 | 0.00 |
| leukocyte activation | 115 | 2.14 | 0.00 | 0.00 |
| cell chemotaxis | 37 | 2.13 | 0.00 | 0.00 |
| lymphocyte activation | 95 | 2.12 | 0.00 | 0.00 |
| regulation of cytokine biosynthetic process | 35 | 2.11 | 0.00 | 0.00 |
| regulation of immune response | 207 | 2.10 | 0.00 | 0.00 |
| granulocyte migration | 15 | 2.09 | 0.00 | 0.00 |
| regulation of response to wounding | 110 | 2.09 | 0.00 | 0.00 |
| positive regulation of response to external stimulus | 75 | 2.08 | 0.00 | 0.00 |
| positive regulation of immune system process | 237 | 2.06 | 0.00 | 0.00 |
| proteinaceous extracellular matrix | 107 | 2.06 | 0.00 | 0.00 |
| innate immune response | 122 | 2.06 | 0.00 | 0.00 |
| regulation of interleukin 6 production | 33 | 2.05 | 0.00 | 0.00 |
| extracellular matrix | 125 | 2.05 | 0.00 | 0.00 |
| extracellular structure organization | 78 | 2.04 | 0.00 | 0.00 |
| defense response | 284 | 2.04 | 0.00 | 0.00 |
| cell activation | 163 | 2.04 | 0.00 | 0.00 |
| leukocyte cell cell adhesion | 75 | 2.02 | 0.00 | 0.00 |
| defense response to bacterium | 44 | 2.00 | 0.00 | 0.00 |
| phagocytosis | 45 | 2.00 | 0.00 | 0.00 |
| positive regulation of cytokine biosynthetic process | 25 | 2.00 | 0.00 | 0.00 |
| extracellular space | 320 | 2.00 | 0.00 | 0.00 |
| regulation of immune system process | 373 | 1.99 | 0.00 | 0.00 |
| single organism cell adhesion | 123 | 1.99 | 0.00 | 0.00 |
| extracellular matrix component | 39 | 1.99 | 0.00 | 0.00 |
| cell activation involved in immune response | 37 | 1.99 | 0.00 | 0.00 |
| growth factor binding | 37 | 1.99 | 0.00 | 0.00 |
| regulation of cell cell adhesion | 115 | 1.98 | 0.00 | 0.00 |
| inflammatory response | 130 | 1.97 | 0.00 | 0.00 |
| leukocyte differentiation | 78 | 1.96 | 0.00 | 0.00 |
| cytokine receptor activity | 25 | 1.96 | 0.00 | 0.00 |
| Negative (33) | | | | |
| ammonium ion binding | 17 | −1.98 | 0.00 | 0.14 |
| organelle inner membrane | 186 | −1.95 | 0.00 | 0.09 |
| mitochondrial matrix | 152 | −1.94 | 0.00 | 0.06 |
| mitochondrial membrane part | 69 | −1.89 | 0.00 | 0.11 |
| mitochondrial protein complex | 62 | −1.86 | 0.00 | 0.13 |
| inner mitochondrial membrane protein complex | 49 | −1.86 | 0.00 | 0.11 |
| mitochondrial part | 323 | −1.85 | 0.00 | 0.11 |
| regulation of postsynaptic membrane potential | 18 | −1.84 | 0.00 | 0.11 |
| mitochondrial respiratory chain complex assembly | 31 | −1.83 | 0.00 | 0.10 |
| respiratory chain | 41 | −1.83 | 0.00 | 0.10 |
| oxidative phosphorylation | 40 | −1.80 | 0.00 | 0.13 |
| mitochondrial translation | 43 | −1.79 | 0.00 | 0.12 |
| ciliary part | 77 | −1.77 | 0.00 | 0.14 |
| regulation of neurotransmitter secretion | 15 | −1.77 | 0.00 | 0.14 |
| mitochondrial respiratory chain complex i biogenesis | 25 | −1.75 | 0.00 | 0.15 |
| mitochondrial envelope | 230 | −1.75 | 0.00 | 0.16 |
| nadh dehydrogenase complex | 24 | −1.74 | 0.00 | 0.16 |
| sodium ion transmembrane transporter activity | 43 | −1.73 | 0.00 | 0.17 |
| thioester biosynthetic process | 18 | −1.72 | 0.01 | 0.17 |
| cilium organization | 45 | −1.71 | 0.00 | 0.18 |
| sodium ion transmembrane transport | 32 | −1.71 | 0.01 | 0.18 |
| cellular respiration | 59 | −1.69 | 0.00 | 0.20 |
| electron transport chain | 44 | −1.68 | 0.01 | 0.21 |
| cellular protein complex disassembly | 42 | −1.68 | 0.01 | 0.21 |
| regulation of neurotransmitter transport | 17 | −1.67 | 0.01 | 0.21 |
| cofactor metabolic process | 105 | −1.67 | 0.00 | 0.21 |
| cell surface receptor signaling pathway involved in cell cell signaling | 22 | −1.67 | 0.01 | 0.20 |
| cilium morphogenesis | 54 | −1.67 | 0.00 | 0.20 |
| mitochondrion organization | 207 | −1.67 | 0.00 | 0.20 |
| microtubule motor activity | 16 | −1.65 | 0.02 | 0.22 |
| fatty acyl coa metabolic process | 19 | −1.64 | 0.02 | 0.23 |
| sodium ion transport | 50 | −1.64 | 0.01 | 0.23 |
| action potential | 31 | −1.63 | 0.01 | 0.24 |

(#) - indicates total number of GO terms that satisfy p <0.05, FDR <0.25

TABLE 7

| | Fibrosis-related | | | | Fibrosis- |
|---|---|---|---|---|---|
| | gene-set: | | | | related gene |
| | present in | | | | set: absent in |
| | organoid and | Organoid | Mouse | | organoid and |
| Category | mouse | LFC | LFC | Category | mouse |
| Cell Adhesion/Migration | ANG | 0.276 | 0.349 | Cell Adhesion/Migration | ADGRE2 |
| Cell Adhesion/Migration | ARF4 | 0.227 | 1.584 | Cell Adhesion/Migration | ADGRG1 |
| Cell Adhesion/Migration | ARPC5 | 0.356 | 1.120 | Cell Adhesion/Migration | AMOTL2A |
| Cell Adhesion/Migration | ARPC5L | 0.053 | −0.094 | Cell Adhesion/Migration | APLNRA |
| Cell Adhesion/Migration | BDKRB1 | 0.616 | 0.064 | Cell Adhesion/Migration | ARC |
| Cell Adhesion/Migration | BTG1 | −0.057 | 1.396 | Cell Adhesion/Migration | ARPC5A |
| Cell Adhesion/Migration | CD151 | 0.220 | −0.152 | Cell Adhesion/Migration | ARPC5B |
| Cell Adhesion/Migration | CD2AP | 0.102 | 0.411 | Cell Adhesion/Migration | ARPC5LA |
| Cell Adhesion/Migration | CD44 | 0.411 | 1.866 | Cell Adhesion/Migration | ARPC5LB |
| Cell Adhesion/Migration | CD47 | 0.242 | 1.014 | Cell Adhesion/Migration | ARPC5LPS1 |
| Cell Adhesion/Migration | CD63 | 0.280 | 1.715 | Cell Adhesion/Migration | ARPIN |
| Cell Adhesion/Migration | CDH2 | −0.062 | −0.931 | Cell Adhesion/Migration | ASAP3 |
| Cell Adhesion/Migration | CLDN1 | 0.354 | −0.091 | Cell Adhesion/Migration | AVL9 |
| Cell Adhesion/Migration | CXCL12 | −0.305 | −0.710 | Cell Adhesion/Migration | AVL9 |
| Cell Adhesion/Migration | CXCR4 | 0.263 | 2.955 | Cell Adhesion/Migration | CASS4 |
| Cell Adhesion/Migration | DDR2 | 0.215 | 0.336 | Cell Adhesion/Migration | CD248 |
| Cell Adhesion/Migration | F11R | −0.261 | −0.257 | Cell Adhesion/Migration | CDH7 |
| Cell Adhesion/Migration | ICAM1 | −0.227 | 0.715 | Cell Adhesion/Migration | CXCR4B |
| Cell Adhesion/Migration | ICAM2 | 0.270 | −0.736 | Cell Adhesion/Migration | DCHS1 |
| Cell Adhesion/Migration | ICAM5 | −0.320 | −0.104 | Cell Adhesion/Migration | DOCK1 |
| Cell Adhesion/Migration | ITGA2 | 0.305 | −0.037 | Cell Adhesion/Migration | ELMO1 |
| Cell Adhesion/Migration | ITGA3 | 0.086 | −0.295 | Cell Adhesion/Migration | ELMO2 |
| Cell Adhesion/Migration | ITGA4 | 0.668 | 1.064 | Cell Adhesion/Migration | ELMO3 |
| Cell Adhesion/Migration | ITGA5 | 0.835 | 1.142 | Cell Adhesion/Migration | FBLIM1 |
| Cell Adhesion/Migration | ITGA6 | −0.183 | 0.610 | Cell Adhesion/Migration | ICAM3 |
| Cell Adhesion/Migration | ITGA7 | 0.015 | −0.558 | Cell Adhesion/Migration | ICAM4 |
| Cell Adhesion/Migration | ITGA8 | −0.016 | −0.165 | Cell Adhesion/Migration | ITGA1 |
| Cell Adhesion/Migration | ITGAE | −0.112 | 0.341 | Cell Adhesion/Migration | ITGA10 |
| Cell Adhesion/Migration | ITGAL | 0.031 | −0.091 | Cell Adhesion/Migration | ITGA11 |
| Cell Adhesion/Migration | ITGAM | −0.099 | 1.746 | Cell Adhesion/Migration | ITGA2B |
| Cell Adhesion/Migration | ITGAV | 0.191 | 1.847 | Cell Adhesion/Migration | ITGA9 |
| Cell Adhesion/Migration | ITGAX | 0.150 | 0.681 | Cell Adhesion/Migration | ITGAD |
| Cell Adhesion/Migration | ITGB1 | 0.338 | 0.776 | Cell Adhesion/Migration | ITGB1B |
| Cell Adhesion/Migration | ITGB1BP1 | 0.008 | −0.210 | Cell Adhesion/Migration | ITGB1BP2 |
| Cell Adhesion/Migration | ITGB2 | −0.058 | 2.423 | Cell Adhesion/Migration | ITGB3BP |
| Cell Adhesion/Migration | ITGB3 | 0.447 | −0.127 | Cell Adhesion/Migration | ITGB6 |
| Cell Adhesion/Migration | ITGB4 | 0.052 | 0.080 | Cell Adhesion/Migration | ITGB6 |
| Cell Adhesion/Migration | ITGB5 | 0.613 | 1.217 | Cell Adhesion/Migration | ITGBL1 |
| Cell Adhesion/Migration | ITGB7 | 0.280 | 0.571 | Cell Adhesion/Migration | MGC127538 |
| Cell Adhesion/Migration | JAM3 | 0.273 | 0.144 | Cell Adhesion/Migration | MYO18A |
| Cell Adhesion/Migration | JUP | −0.109 | −0.981 | Cell Adhesion/Migration | NCU02750 |
| Cell Adhesion/Migration | L1CAM | 0.462 | −0.316 | Cell Adhesion/Migration | NDNF |
| Cell Adhesion/Migration | PIK3CA | 0.019 | −0.297 | Cell Adhesion/Migration | P130CAS |
| Cell Adhesion/Migration | PIK3CD | 0.046 | 0.024 | Cell Adhesion/Migration | PALLD |
| Cell Adhesion/Migration | RAC1 | 0.049 | 0.309 | Cell Adhesion/Migration | PIK3CB |
| Cell Adhesion/Migration | RHOA | 0.265 | 0.581 | Cell Adhesion/Migration | PIK3CG |
| Cell Adhesion/Migration | RHOB | 0.557 | 0.013 | Cell Adhesion/Migration | RAC5 |
| Cell Adhesion/Migration | RHOC | 0.291 | 0.689 | Cell Adhesion/Migration | RAC9 |
| Cell Adhesion/Migration | RHOD | 0.038 | 0.537 | Cell Adhesion/Migration | RHO1 |
| Cell Adhesion/Migration | RHOG | 0.246 | 0.710 | Cell Adhesion/Migration | RHO5 |
| Cell Adhesion/Migration | RHOH | 0.025 | 0.237 | Cell Adhesion/Migration | RHOBTB3 |
| Cell Adhesion/Migration | RHOJ | 0.206 | 0.669 | Cell Adhesion/Migration | RHOF |
| Cell Adhesion/Migration | RHOQ | −0.137 | 0.148 | Cell Adhesion/Migration | RHOL |
| Cell Adhesion/Migration | RHOU | 0.226 | 0.811 | Cell Adhesion/Migration | RHOV |
| Cell Adhesion/Migration | RND2 | −0.134 | 0.056 | Cell Adhesion/Migration | RND1 |
| Cell Adhesion/Migration | RPS6KB1 | 0.037 | −0.114 | Cell Adhesion/Migration | RND3 |
| Cell Adhesion/Migration | SDC1 | 0.940 | 0.563 | Cell Adhesion/Migration | ROP1 |
| Cell Adhesion/Migration | SDC2 | 0.330 | 0.529 | Cell Adhesion/Migration | ROP10 |
| Cell Adhesion/Migration | SDC3 | 0.191 | −0.129 | Cell Adhesion/Migration | ROP2 |
| Cell Adhesion/Migration | SDC4 | −0.018 | 1.028 | Cell Adhesion/Migration | ROP9 |
| Cell Adhesion/Migration | TPM3 | 0.390 | 0.671 | Cell Adhesion/Migration | SDC |
| Extracellular Matrix | ABI1 | 0.165 | −0.014 | Cell Adhesion/Migration | SDN1 |
| Extracellular Matrix | ACAN | 0.220 | 0.071 | Extracellular Matrix | ABI1 |
| Extracellular Matrix | BCAN | 0.333 | −0.066 | Extracellular Matrix | ABI3BP |
| Extracellular Matrix | BGN | 0.257 | 2.460 | Extracellular Matrix | CCDC88AA |
| Extracellular Matrix | CCDC80 | 0.755 | 1.936 | Extracellular Matrix | CCDC88AB |
| Extracellular Matrix | CCDC88A | −0.115 | 2.054 | Extracellular Matrix | COL16A1 |
| Extracellular Matrix | COL10A1 | 0.246 | −0.143 | Extracellular Matrix | COL20A1 |
| Extracellular Matrix | COL11A1 | 0.465 | 3.029 | Extracellular Matrix | COL21A1 |
| Extracellular Matrix | COL11A2 | −0.852 | −0.276 | Extracellular Matrix | COL23A1 |
| Extracellular Matrix | COL12A1 | 0.146 | 0.004 | Extracellular Matrix | COL24A1 |
| Extracellular Matrix | COL13A1 | 0.333 | −0.165 | Extracellular Matrix | COL25A1 |
| Extracellular Matrix | COL14A1 | −0.249 | 3.304 | Extracellular Matrix | COL27A1 |
| Extracellular Matrix | COL15A1 | 0.315 | 1.737 | Extracellular Matrix | COL28A1 |
| Extracellular Matrix | COL17A1 | 0.099 | −0.308 | Extracellular Matrix | COL4A3BP |
| Extracellular Matrix | COL18A1 | 0.030 | 1.139 | Extracellular Matrix | COL4A6 |
| Extracellular Matrix | COL19A1 | −0.300 | 0.042 | Extracellular Matrix | COL5A3 |
| Extracellular Matrix | COL1A1 | 0.808 | 3.966 | Extracellular Matrix | COL6A4P1 |
| Extracellular Matrix | COL1A2 | 0.627 | 3.796 | Extracellular Matrix | COL6A4P2 |
| Extracellular Matrix | COL22A1 | −1.172 | 0.043 | Extracellular Matrix | COL6A5 |
| Extracellular Matrix | COL26A1 | −0.272 | −0.107 | Extracellular Matrix | COL6A6 |
| Extracellular Matrix | COL2A1 | −0.388 | −0.025 | Extracellular Matrix | COL8A2 |
| Extracellular Matrix | COL3A1 | 0.229 | 3.449 | Extracellular Matrix | COMP |
| Extracellular Matrix | COL4A1 | −0.027 | 1.426 | Extracellular Matrix | CTHRC1 |
| Extracellular Matrix | COL4A2 | −0.074 | 0.705 | Extracellular Matrix | ECM2 |
| Extracellular Matrix | COL4A3 | 0.023 | −0.283 | Extracellular Matrix | EGFLAM |
| Extracellular Matrix | COL4A4 | −0.015 | 0.110 | Extracellular Matrix | FNDC3B |
| Extracellular Matrix | COL4A5 | −0.276 | 0.361 | Extracellular Matrix | FSCN1A |
| Extracellular Matrix | COL5A1 | 0.681 | 2.920 | Extracellular Matrix | FSCN1B |
| Extracellular Matrix | COL5A2 | 0.572 | 4.404 | Extracellular Matrix | FSCN2 |
| Extracellular Matrix | COL6A1 | 0.515 | 1.845 | Extracellular Matrix | FSCN2A |
| Extracellular Matrix | COL6A2 | 0.478 | 1.742 | Extracellular Matrix | FSCN2B |
| Extracellular Matrix | COL6A3 | 0.314 | 2.564 | Extracellular Matrix | FSCN3 |
| Extracellular Matrix | COL7A1 | 0.236 | 0.370 | Extracellular Matrix | GPC6 |
| Extracellular Matrix | COL8A1 | 0.604 | 3.354 | Extracellular Matrix | LAMB2P1 |
| Extracellular Matrix | COL9A1 | −0.419 | 0.653 | Extracellular Matrix | LAMB4 |
| Extracellular Matrix | COL9A2 | −0.504 | −0.063 | Extracellular Matrix | LAMC3 |
| Extracellular Matrix | COL9A3 | −0.245 | 0.067 | Extracellular Matrix | LTBP2 |
| Extracellular Matrix | DCN | 0.332 | 0.234 | Extracellular Matrix | PXDN |
| Extracellular Matrix | DPT | 0.602 | 0.716 | Extracellular Matrix | SULF1 |
| Extracellular Matrix | FBLN1 | 0.206 | 0.379 | Extracellular Matrix | THBS1 |
| Extracellular Matrix | FBN1 | 0.820 | 3.370 | Extracellular Matrix | THSD4 |
| Extracellular Matrix | FBN2 | −0.403 | 0.580 | Extracellular Matrix | TNN |
| Extracellular Matrix | FN1 | 0.368 | 1.595 | Extracellular Matrix | TNS3 |
| Extracellular Matrix | FSCN1 | 0.658 | 0.788 | Extracellular Matrix | VWA1 |
| Extracellular Matrix | HAPLN1 | 0.424 | −0.364 | Growth Factor | DRK |
| Extracellular Matrix | HAS1 | 0.528 | 0.375 | Growth Factor | FGF16 |
| Extracellular Matrix | HAS2 | 1.153 | 0.577 | Growth Factor | FGF17 |
| Extracellular Matrix | IBSP | 0.508 | 0.061 | Growth Factor | FGF19 |
| Extracellular Matrix | LAMA1 | 0.806 | −0.104 | Growth Factor | FGF20 |
| Extracellular Matrix | LAMA2 | −0.144 | −0.621 | Growth Factor | FGF21 |
| Extracellular Matrix | LAMA3 | −0.400 | −0.164 | Growth Factor | FGF22 |
| Extracellular Matrix | LAMA4 | 0.206 | 0.745 | Growth Factor | FGF23 |
| Extracellular Matrix | LAMA5 | −0.356 | −0.971 | Growth Factor | FGF3 |
| Extracellular Matrix | LAMB1 | 0.141 | 0.801 | Growth Factor | FGF4 |
| Extracellular Matrix | LAMB2 | −0.319 | −1.289 | Growth Factor | FGFBP1 |
| Extracellular Matrix | LAMB3 | −0.364 | −1.129 | Growth Factor | FGFBP2 |
| Extracellular Matrix | LAMC1 | 0.036 | 0.169 | Growth Factor | FGFBP3 |
| Extracellular Matrix | LAMC2 | −0.120 | −0.138 | Growth Factor | FGFRL1 |
| Extracellular Matrix | LCP1 | 0.915 | 2.635 | Growth Factor | TGFBR3L |
| Extracellular Matrix | LOX | 0.836 | 5.156 | Growth Factor | TGFBRAP1 |
| Extracellular Matrix | LOXL1 | 0.908 | 1.097 | Protease/Inhibitor | ADAMTS12 |
| Extracellular Matrix | LOXL2 | 0.653 | 3.905 | Protease/Inhibitor | ADAMTS14 |
| Extracellular Matrix | MATN1 | 0.405 | 0.001 | Protease/Inhibitor | ADAMTS2 |
| Extracellular Matrix | MATN3 | 0.353 | 0.160 | Protease/Inhibitor | ADAMTS4 |
| Extracellular Matrix | MFAP2 | 0.588 | 1.858 | Protease/Inhibitor | ADAMTSL4 |
| Extracellular Matrix | MFAP5 | 1.205 | 3.319 | Protease/Inhibitor | BAMBI |
| Extracellular Matrix | P4HA1 | 0.500 | 0.870 | Protease/Inhibitor | CST20 |
| Extracellular Matrix | PLOD3 | 0.449 | 0.778 | Protease/Inhibitor | MMP1 |
| Extracellular Matrix | POSTN | 0.671 | 4.651 | Protease/Inhibitor | MMP19 |
| Extracellular Matrix | RELN | 0.707 | −0.205 | Protease/Inhibitor | MMP20 |
| Extracellular Matrix | SMOC2 | −0.396 | −0.011 | Protease/Inhibitor | MMP21 |
| Extracellular Matrix | SPARC | 0.468 | 2.554 | Protease/Inhibitor | MMP23A |
| Extracellular Matrix | SPOCK2 | 0.330 | −0.314 | Protease/Inhibitor | MMP23B |
| Extracellular Matrix | SPP1 | 0.661 | 6.666 | Protease/Inhibitor | MMP25 |
| Extracellular Matrix | TNC | 0.202 | 4.635 | Protease/Inhibitor | MMP26 |
| Extracellular Matrix | TNXB | 1.080 | −1.217 | Protease/Inhibitor | MMP27 |
| Extracellular Matrix | VCAN | 0.564 | 2.425 | Protease/Inhibitor | MMP28 |
| Extracellular Matrix | VTN | 0.246 | −0.765 | Protease/Inhibitor | SPINK5 |
| Growth Factor | CTGF | 0.536 | 2.397 | Protease/Inhibitor | TIMP4 |
| Growth Factor | FGF1 | −0.235 | −1.174 | Protease/Inhibitor | TLL2 |
| Growth Factor | FGF10 | 0.485 | −0.127 | | |
| Growth Factor | FGF11 | 0.203 | −0.207 | | |
| Growth Factor | FGF12 | −0.703 | −0.080 | | |
| Growth Factor | FGF13 | −0.289 | −1.617 | | |
| Growth Factor | FGF14 | 0.182 | −0.141 | | |
| Growth Factor | FGF18 | −0.316 | −0.154 | | |
| Growth Factor | FGF2 | −0.378 | −0.106 | | |
| Growth Factor | FGF5 | 0.602 | −0.314 | | |
| Growth Factor | FGF6 | −0.016 | −0.495 | | |
| Growth Factor | FGF7 | −0.438 | 0.401 | | |
| Growth Factor | FGF8 | 0.001 | −0.109 | | |
| Growth Factor | FGF9 | −0.400 | −0.698 | | |
| Growth Factor | FGFR1 | 0.200 | 1.204 | | |
| Growth Factor | FGFR2 | 0.231 | 0.021 | | |
| Growth Factor | FGFR3 | 0.206 | 0.224 | | |
| Growth Factor | FGFR4 | −0.020 | −0.231 | | |
| Growth Factor | FLT1 | −0.399 | −0.484 | | |
| Growth Factor | HBEGF | −0.215 | 0.111 | | |
| Growth Factor | KDR | 0.132 | −1.107 | | |
| Growth Factor | MDK | −0.210 | 0.531 | | |
| Growth Factor | PDGFB | −0.342 | −0.328 | | |
| Growth Factor | PDGFRA | −0.022 | 0.991 | | |
| Growth Factor | PDGFRB | 0.216 | 0.306 | | |
| Growth Factor | SMAD3 | −0.338 | 0.022 | | |
| Growth Factor | TGFB1 | 0.629 | 0.608 | | |
| Growth Factor | TGFB1I1 | 0.187 | 0.913 | | |
| Growth Factor | TGFB2 | −0.121 | 1.581 | | |
| Growth Factor | TGFB3 | −0.167 | 1.687 | | |
| Growth Factor | TGFBI | 1.142 | 3.291 | | |
| Growth Factor | TGFBR1 | 0.564 | 0.710 | | |
| Growth Factor | TGFBR2 | 0.037 | −0.381 | | |
| Growth Factor | TGFBR3 | −0.058 | −0.409 | | |
| Growth Factor | TGIF1 | 0.278 | 2.251 | | |
| Growth Factor | TGIF2 | 0.018 | 0.156 | | |
| Protease/Inhibitor | A2M | −0.432 | −0.176 | | |
| Protease/Inhibitor | ADAM15 | 0.239 | −0.167 | | |
| Protease/Inhibitor | ADAM9 | 0.100 | 2.069 | | |
| Protease/Inhibitor | BMP1 | 0.551 | 1.364 | | |
| Protease/Inhibitor | CAPN2 | 0.249 | 1.033 | | |
| Protease/Inhibitor | CST3 | 0.378 | 0.472 | | |
| Protease/Inhibitor | CTSK | 1.356 | 2.917 | | |
| Protease/Inhibitor | CTSL | 0.214 | 1.823 | | |
| Protease/Inhibitor | CTSS | −0.441 | 4.086 | | |
| Protease/Inhibitor | DPP4 | 0.310 | 0.054 | | |
| Protease/Inhibitor | FAP | 0.989 | 2.330 | | |
| Protease/Inhibitor | GREM1 | 0.509 | −0.230 | | |
| Protease/Inhibitor | HPN | −0.472 | −0.014 | | |
| Protease/Inhibitor | HTRA1 | 0.267 | −0.079 | | |
| Protease/Inhibitor | MMP10 | 0.567 | 0.102 | | |
| Protease/Inhibitor | MMP11 | 0.634 | 0.434 | | |
| Protease/Inhibitor | MMP12 | 0.019 | 3.776 | | |
| Protease/Inhibitor | MMP13 | −0.484 | 0.025 | | |
| Protease/Inhibitor | MMP14 | 0.982 | 3.110 | | |
| Protease/Inhibitor | MMP15 | 0.175 | −1.789 | | |
| Protease/Inhibitor | MMP16 | 0.710 | 0.349 | | |
| Protease/Inhibitor | MMP17 | 0.865 | 0.245 | | |
| Protease/Inhibitor | MMP2 | 0.946 | −0.118 | | |
| Protease/Inhibitor | MMP24 | 0.218 | −0.277 | | |
| Protease/Inhibitor | MMP3 | 0.048 | 0.999 | | |
| Protease/Inhibitor | MMP7 | −0.016 | −0.118 | | |
| Protease/Inhibitor | MMP8 | 0.051 | 0.263 | | |
| Protease/Inhibitor | MMP9 | −0.308 | 2.068 | | |
| Protease/Inhibitor | SERPINE1 | 0.637 | 2.126 | | |
| Protease/Inhibitor | SERPINH1 | 0.430 | 1.416 | | |
| Protease/Inhibitor | SPINT1 | −0.560 | −0.013 | | |
| Protease/Inhibitor | TIMP2 | 0.356 | 1.057 | | |
| Protease/Inhibitor | TIMP3 | 0.441 | −0.113 | | |

TABLE 8

| Calcium | | | Calcium |
|---|---|---|---|
| handling-related | | | handling- |
| gene set: | | | related gene |
| present in | | | set: absent in |
| organoid and | Organoid | Mouse | organoid and |
| mouse | LFC | LFC | mouse |
| ADCY2 | 0.068 | 0.006 | ADCY1 |
| ADCY3 | −0.032 | −0.054 | ADRA1D |
| ADCY4 | 0.132 | −0.269 | AGTR1 |
| ADCY7 | 0.449 | 2.352 | ATP2B1 |
| ADCY8 | 1.011 | −0.009 | ATB2B3 |
| ADCY9 | −0/212 | −1.361 | ATP2B4 |
| ADORA2A | −0.038 | −0.338 | AVPR1B |
| ADORA2B | 0.465 | 0.747 | CACNA1D |
| ADRA1A | 0.029 | −0.744 | CACNA1F |
| ADRA1B | 0.818 | −0.947 | CACNA1I |
| ADRB1 | −0.835 | −0.862 | CALM3 |
| ADRB2 | −0.522 | 0.428 | CALML3 |
| ADRB3 | 0.883 | −0.088 | CALML5 |
| ATP2A1 | −0.447 | 0.101 | CALML6 |
| ATP2A2 | −0.603 | −2.245 | CAMK2G |
| ATP2A3 | 0.133 | −0.210 | CHRM2 |
| ATP2B2 | −0.346 | −0.233 | CHRM3 |
| AVPR1A | 0.140 | 0.058 | CHRM5 |
| BDKRB1 | 0.618 | 0.064 | CYSLTR1 |
| BDKRB2 | 0.095 | −0.236 | CYSLTR2 |
| CACNA1A | 0.739 | −0.201 | DRD1 |
| CACNA1B | 0.147 | −0.076 | DRD5 |
| CACNA1C | 0.322 | −2.161 | EDNRA |
| CACNA1E | 0.558 | −0.120 | ERBB2 |
| CACNA1G | −0.952 | −0.808 | GNAL |
| CACNA1H | 0.005 | −0.307 | GRM1 |
| CACNA1S | −0.294 | −1.115 | GRM5 |
| CALM1 | 0.325 | 0.895 | HTR2A |
| CALM2 | 0.038 | 0.229 | HTR6 |
| CALML4 | −0.150 | 1.437 | IGH |
| CAMK2A | −0.0442 | −0.495 | ITPKA |
| CAMK2B | −0.316 | −0.727 | ITPKC |
| CAMK2D | −0.253 | −0.780 | ITPR3 |
| CAMK4 | 0.385 | −0.026 | LTBR2 |
| CCKAR | 0.034 | −0.154 | MCU |
| CCKBR | −0.048 | −0.246 | MYLK2 |
| CD38 | 0.034 | 0.492 | MYLK3 |
| CHRM1 | −0.905 | −0.029 | MYLK4 |
| CHRNA7 | −0.118 | 0.133 | ORAI1 |
| EDNRB | −0.917 | 0.448 | ORAI2 |
| EGFR | −0.194 | 0.537 | ORAI3 |
| ERBB3 | −0.610 | −0.904 | OXTR |
| ERBB4 | −0.641 | −0.428 | P2RX2 |
| F2R | 0.533 | 2.125 | P2RX3 |
| GNA11 | 0.094 | −0.260 | P2RX5 |
| GNA14 | 0.134 | 0.249 | PLCB2 |
| GNA15 | 0.000 | 0.070 | PLCB4 |
| GNAQ | 0.324 | 0.146 | PLCD3 |
| GNAS | 0.013 | −0.132 | PLCD4 |
| GRIN1 | −0.030 | 0.018 | PLCE1 |
| GRIN2A | −0.152 | −0.330 | PLCZ1 |
| GRIN2C | −0.219 | −0.015 | PLN |
| GRIN2D | 0.317 | −0.039 | PPP3R2 |
| GRPR | −0.083 | −0.119 | PRKACG |
| HRH1 | 0.379 | −0.073 | SLC25A31 |
| HRH2 | −0.371 | −0.124 | SLC25A6 |
| HTR2B | 0.199 | 0.981 | SLC8A2 |
| HTR2C | −0.019 | −0.253 | SLC8A3 |
| HTR4 | −0.0331 | −0.022 | STIM2 |
| HTR5A | 0.015 | 0.034 | TACR3 |
| HTR7 | 0.196 | 0.103 | TRHR |
| ITPKB | 0.340 | −0.724 | |
| ITPR1 | 0.386 | 0.397 | |
| ITPR2 | 0.164 | 0.111 | |
| LHCGR | 0.054 | −0.075 | |
| MYLK | −0.013 | 0.801 | |
| NOS1 | −0.062 | −0.283 | |
| NOS2 | −0.155 | −0.284 | |
| NOS3 | −0.020 | −0.722 | |
| NTSR1 | −0.031 | −0.117 | |
| P2RX1 | −0.258 | −0.047 | |
| P2RX4 | 0.386 | 1.795 | |
| P2RX6 | −0.147 | −0.249 | |
| P2RX7 | −0.500 | 0.093 | |
| PDE1A | 0.040 | −0.830 | |
| PDE1B | 0.323 | 0.438 | |
| PDE1C | −0.342 | −0.322 | |
| PDGFRA | −0.022 | 0.991 | |
| PDGFRB | 0.216 | 0.306 | |
| PHKA1 | −0.361 | −0.988 | |
| PHKA2 | −0.244 | −0.413 | |
| PHKB | −0.169 | −0.181 | |
| PHKG1 | −0.350 | −1.340 | |
| PHKG2 | −0.131 | 0.128 | |
| PLCB1 | −0.466 | 0.351 | |
| PLCB3 | 0.079 | −0.095 | |
| PLCD1 | −0.154 | −0.349 | |
| PLCG1 | −0.006 | −0.059 | |
| PLCG2 | −0.706 | 0.501 | |
| PPIF | −0.213 | −2.554 | |
| PPP3CA | 0.214 | 0.664 | |
| PPP3CB | −0.021 | −0.393 | |
| PPP3CC | 0.173 | −1.066 | |
| PPP3R1 | 0.040 | −0.101 | |
| PRKACA | −0.248 | −1.260 | |
| PRKACB | 0.030 | 0.204 | |
| PRKCA | 0.308 | −0.144 | |
| PRKCB | −0.100 | 1.689 | |
| PRKCG | 0.119 | −0.186 | |
| PTAFR | 0.037 | 1.176 | |
| PTGER1 | 0.021 | −0.136 | |
| PTGER3 | 0.387 | 0.182 | |
| PTGFR | 0.016 | −0.499 | |
| PTK2B | −0.200 | 1.121 | |
| RYR1 | 0.023 | 0.119 | |
| RYR2 | −0.416 | −2.351 | |
| RYR3 | −0.612 | −0.137 | |
| SLC25A4 | −0.074 | −1.062 | |
| SLC25A5 | −0.295 | −0.240 | |
| SLC8A1 | −0.311 | −1.085 | |
| SPHK1 | 0.689 | 1.100 | |
| SPHK2 | 0.269 | −0.180 | |
| STIM1 | 0.131 | 0.240 | |
| TACR1 | 0.021 | −0.134 | |
| TACR2 | 0.078 | −0.132 | |
| TBXA2R | −0.256 | 0.173 | |
| TNNC1 | −0.012 | −1.547 | |
| TNNC2 | −0.132 | 0.211 | |
| VDAC1 | −0.132 | −0.275 | |
| VDAC2 | −0.178 | −0.451 | |
| VDAC3 | −0.197 | −1.138 | |

### REFERENCES CITED IN BACKGROUND, SUMMARY, AND EXAMPLE 10

1 Clevers, H. Modeling Development and Disease with Organoids. Cell 165, 1586-1597, doi:10.1016/j.cell.2016.05.082 (2016).
2 Fatehullah, A., Tan, S. H. & Barker, N. Organoids as an in vitro model of human development and disease. Nature cell biology 18, 246-254, doi:10.1038/ncb3312 (2016).
3 Leite, S. B. et al. Novel human hepatic organoid model enables testing of drug-induced liver fibrosis in vitro. Biomaterials 78, 1-10, doi:10.1016/j.biomaterials.2015.11.026 (2016).
4 Voges, H. K. et al. Development of a human cardiac organoid injury model reveals innate regenerative potential. Development 144, 1118-1127, doi:10.1242/dev.143966 (2017).
5 Benjamin, E. J. et al. Heart Disease and Stroke Statistics-2017 Update: A Report From the American Heart Association. Circulation 135, e146-e603, doi:10.1161/CIR.0000000000000485 (2017).
6 Horvath, P. et al. Screening out irrelevant cell-based models of disease. Nat Rev Drug Discov 15, 751-769, doi:10.1038/nrd.2016.175 (2016).
7 Gheorghiade, M. et al. Developing New Treatments for Heart Failure: Focus on the Heart. Circulation. Heart failure 9, doi:10.1161/CIRCHEARTFAILURE.115.002727 (2016).
8 Fine, B. & Vunjak-Novakovic, G. Shortcomings of Animal Models and the Rise of Engineered Human Cardiac Tissue. Acs Biomater Sci Eng 3, 1884-1897, doi:10.1021/acsbiomaterials.6b00662 (2017).
9 Kaye, D. M. & Krum, H. Drug discovery for heart failure: a new era or the end of the pipeline? Nat Rev Drug Discov 6, 127-139, doi:10.1038/nrd2219 (2007).
10 Radisic, M. et al. Oxygen gradients correlate with cell density and cell viability in engineered cardiac tissue. Biotechnology and bioengineering 93, 332-343, doi:10.1002/bit.20722 (2006).
11 Richards, D. J. et al. Inspiration from heart development: Biomimetic development of functional human cardiac organoids. Biomaterials 142, 112-123, doi:10.1016/j.biomaterials.2017.07.021 (2017).
12 Frangogiannis, N. G. Pathophysiology of Myocardial Infarction. Compr Physiol 5, 1841-1875, doi:10.1002/cphy.c150006 (2015).
13 Lymperopoulos, A., Rengo, G. & Koch, W. J. Adrenergic nervous system in heart failure: pathophysiology and therapy. Circulation research 113, 739-753, doi:10.1161/CIRCRESAHA.113.300308 (2013).
14 Brown, D. A. et al. Analysis of oxygen transport in a diffusion-limited model of engineered heart tissue. Biotechnology and bioengineering 97, 962-975, doi:10.1002/bit.21295 (2007).
15 Davis, B. H. et al. Effects of myocardial infarction on the distribution and transport of nutrients and oxygen in porcine myocardium. Journal of biomechanical engineering 134, 101005, doi:10.1115/1.4007455 (2012).
16 Semenza, G. L. Hypoxia-inducible factor 1: regulator of mitochondrial metabolism and mediator of ischemic preconditioning. Biochimica et biophysica acta 1813, 1263-1268, doi:10.1016/j.bbamcr.2010.08.006 (2011).
17 Chen, W. & Frangogiannis, N. G. Fibroblasts in post-infarction inflammation and cardiac repair. Biochimica et biophysica acta 1833, 945-953, doi:10.1016/j.bbamcr.2012.08.023 (2013).
18 Trickey, W. R., Lee, G. M. & Guilak, F. Viscoelastic properties of chondrocytes from normal and osteoarthritic human cartilage. J Orthop Res 18, 891-898, doi:10.1002/jor.1100180607 (2000).
19 Richardson, W. J., Clarke, S. A., Quinn, T. A. & Holmes, J. W. Physiological Implications of Myocardial Scar Structure. Compr Physiol 5, 1877-1909, doi:10.1002/cphy.c140067 (2015).
20 van Spreeuwel, A. C. C. et al. Mimicking Cardiac Fibrosis in a Dish: Fibroblast Density Rather than Collagen Density Weakens Cardiomyocyte Function. J Cardiovasc Transl Res 10, 116-127, doi:10.1007/s12265-017-9737-1 (2017).
21 Duan, Q. et al. BET bromodomain inhibition suppresses innate inflammatory and profibrotic transcriptional networks in heart failure. Science translational medicine 9, doi:10.1126/scitranslmed.aah5084 (2017).
22 Sadeghi, A. H. et al. Engineered 3D Cardiac Fibrotic Tissue to Study Fibrotic Remodeling. Advanced healthcare materials 6, doi:10.1002/adhm.201601434 (2017).
23 Ugolini, G. S. et al. Human cardiac fibroblasts adaptive responses to controlled combined mechanical strain and oxygen changes in vitro. eLife 6, doi:10.7554/eLife.22847 (2017).
24 Occhetta, P. et al. A three-dimensional in vitro dynamic micro-tissue model of cardiac scar formation. Integr Biol (Camb) 10, 174-183, doi:10.1039/c7ib00199a (2018).
25 Lou, Q., Janardhan, A. & Efimov, I. R. Remodeling of calcium handling in human heart failure. Adv Exp Med Biol 740, 1145-1174, doi:10.1007/978-94-007-2888-2_52 (2012).
26 Bhar-Amato, J., Davies, W. & Agarwal, S. Ventricular Arrhythmia after Acute Myocardial Infarction: ‘The Perfect Storm’. Arrhythm Electrophysiol Rev 6, 134-139, doi:10.15420/aer.2017.24.1 (2017).
27 Huisken, J. & Stainier, D. Y. Selective plane illumination microscopy techniques in developmental biology. Development 136, 1963-1975, doi:10.1242/dev.022426 (2009).
28 Lowes, B. D. et al. Serial gene expression profiling in the intact human heart. J Heart Lung Transplant 25, 579-588, doi:10.1016/j.healun.2006.01.006 (2006).
29 Stevens, K. R. & Murry, C. E. Human Pluripotent Stem Cell-Derived Engineered Tissues: Clinical Considerations. Cell stem cell 22, 294-297, doi:10.1016/j.stem.2018.01.015 (2018).

1. A three-dimensional (3D) myocardial infarct organoid, comprising cardiomyocytes and non-myocytes, wherein the 3D myocardial infarct organoid comprises:
(a) an apoptotic interior region due to lack of oxygen surrounded by a viable periphery that comprises, consists essentially of, or consists of a region of about 20 μm to about 75 μm from the organoid edge;
(b) a ratio of a terminal deoxynucleotidyl transferase dUTP nick end labeling (TUNEL)-positive area to a 4′,6-diamidino-2-phenylindole (DAPI)-positive area ratio in the apoptotic interior region of in a organoid cross-section ranging from about 0.03 to about 1.0;
(c) a contraction amplitude from about 0% to about 5%;
(d) a beat rate of about 0 to 90 beats per minute;
(e) a calcium transient amplitude measured as a change in fluorescence divided by the starting fluorescence of about 0% to about 40%;
(f) upregulated or downregulated fibrosis-related genes, wherein the downregulated genes comprise COL22A1, COL11A2, FGF12, SPINT1, COL9A2, MMP13, HPN, CTSS, FGF7, A2M, COL9A1, FBN2, FGF9, LAMA3, FLT1, SMOC2, COL2A1, FGF2, LAMB3, LAMAS, PDGFB, SMAD3, ICAM5, LAMB2, FGF18, MMP9, CXCL12, COL19A1, FGF13, COL4A5, COL26A1, F11R, COL14A1, COL9A3, FGF1, ICAM1, HBEGF, MDK, ITGA6, TGFB3, LAMA2, RHOQ, RND2, TGFB2, LAMC2, CCDC88A, ITGAE, JUP, ITGAM, COL4A2, CDH2, ITGB2, TGFBR3, BTG1, COL4A1, COL4A3, PDGFRA, FGFR4, SDC4, MMPI, FGF6, ITGA8, and/or COL4A4,
and upregulated genes comprise FGF8, ITGB1BP1, ITGA7, TGIF2, MMP12, PIK3CA, RHOH, COL18A1, ITGAL, LAMC1, RPS6KB1, TGFBR2, RHOD, PIK3CD, MMP3, RAC1, MMP8, ITGB4, ARPC5L, ITGA3, COL17A1, ADAM9 , CD2AP, KDR, LAMB1, COL12A1, ITGAX, ABI1, MMP15, FGF14, TGFB1I1, SDC3, ITGAV, FGFR1, TNC, FGF11, FGFR3, RHOJ, LAMA4, FBLN1, CTSL, DDR2, PDGFRB, MMP24, CD151, ACAN, RHOU, ARF4, COL3A1, FGFR2, COL7A1, ADAM15, CD47, COL10A1, VTN, RHOG, CAPN2, BGN, CXCR4, HTRA1, ICAM2, JAM3, ANG, TGIF1, ITGB7, CD63, RHOA, RHOC, ITGA2, DPP4, COL6A3, COL15A1, SDC2, SPOCK2, DCN, BCAN, COL13A1, ITGB1, MATN3, CLDN1, TIMP2, ARPC5, FN1, CST3, TPM3, MATN1, CD44, HAPLN1, SERPINH1, TIMP3, ITGB3, PLOD3, L1CAM, COL11A1, SPARC, COL6A2, FGF10, P4HA1, IBSP, GREM1, COL6A1, HAS1, CTGF, BMP1, RHOB, VCAN, TGFBR1, MMP10, COL5A2, MFAP2, FGF5, DPT, COL8A1, ITGB5, BDKRB1, COL1A2, TGFB1, MMP11, SERPINE1, LOXL2, FSCN1, SPP1, ITGA4, POSTN, COL5A1, RELN, MMP16, CCDC80, LAMA1, COL1A1, FBN1, ITGA5, LOX, MMP17, LOXL1, LCP1, SDC1, MMP2, MMP14, FAP, TNXB, TGFBI, HAS2, MFAP5, and/or CTSK;
(g) upregulated or downregulated calcium signaling-related genes comprising genes from the Kyoto Encyclopedia of Genes and Genomes (KEGG) calcium signaling pathway, wherein the downregulated genes comprise CACNA1G, EDNRB, CHRM1, ADRB1, PLCG2, ERBB4, RYR3, ERBB3, ATP2A2, ADRB2, P2RX7, PLCB1, ATP2A1, CAMK2A, RYR2, HRH2, PHKA1, PHKG1, ATP2B2, PDE1C, HTR4, CACNA1C, CAMK2B, SLC8A1, SLC25A5, CACNA1S, P2RX1, TBXA2R, CAMK2D, PRKACA, PHKA2, GRIN2C, PPIF, ADCY9, PTK2B, VDAC3, EGFR, VDAC2, PHKB, NOS2, PLCD1, GRIN2A, CALML4, P2RX6, TNNC2, VDAC1, PHKG2, CHRNA7, PRKCB, GRPR, SLC25A4, NOS1, CCKBR, ADORA2A, ADCY3, NTSR1, GRIN1, ADRA1A, PDGFRA, PPP3CB, NOS3, HTR2C, MYLK, TNNC1, and/or PLCG,
and the upregulated genes comprise GNA15, CACNA1H, GNAS, HTR5A, PTGFR, PTGER1, TACR1, RYR1, PRKACB, CCKAR, CD38, PTAFR, CALM2, PDE1A, PPP3R1, LHCGR, ADCY2, TACR2, PLCB3, GNA11, BDKRB2, PRKCG, STIM1, ADCY4, ATP2A3, GNA14, AVPR1A, CACNA1B, ITPR2, PPP3CC, HTR7, HTR2B, PPP3CA, PDGFRB, SPHK2, PRKCA, GRIN2D, PDE1B, GNAQ, CALM1, ITPKB, HRH1, CAMK4, P2RX4, PTGER3, ITPR1, ADCY7, ADORA2B, F2R, CACNA1E, BDKRB1, SPHK1, CACNA1A, ADRA1B, ADRB3, ITPR3, and/or ADCY8; and/or
(h) an elastic modulus of about 3 kPa to about 5 kPa.

2. The 3D myocardial infarct organoid of claim 1, wherein the 3D myocardial infarct organoid beats asynchronously.

3. The 3D myocardial infarct organoid of claim 1, wherein the cardiomyocytes comprise pluripotent stem cell-derived cardiomyocytes (PSC-CMs), cardiac progenitor cells, primary cardiomyocytes, or any combination thereof.

4. The 3D myocardial infarct organoid of claim 1, wherein the cardiomyocytes and non-myocytes are present in a ratio of about 95:5 to about 5:95 of cardiomyocytes to non-myocytes.

5. The 3D myocardial infarct organoid of claim 4, wherein the cardiomyocytes and non-myocytes are present in a ratio of about 60:40 to about 40:60 of cardiomyocytes to non-myocytes.

6. The 3D myocardial infarct organoid of claim 1, wherein the non-myocytes comprise fibroblasts (FBs), endothelial cells (ECs), and mesenchymal stem cells (MSCs), or any combination thereof.

7. The 3D myocardial infarct organoid of claim 6, wherein the non-myocytes comprise FBs in amount of about 50% to 60% based on the total number of non-myocytes, ECs in an amount of about 25% to about 35% based on the total number of non-myocytes, and MSCs in an amount of about 10% to about 20% based on the total number of non-myocytes.

8. The 3D myocardial infarct organoid of claim 1, wherein the cardiomyocytes and/or the non-myocytes are from a human.

9. A method of making a 3D myocardial infarct organoid, the method comprising:
culturing cardiomyocytes with non-myocytes for about 1 day to 20 days to form a self-assembled 3D cardiac organoid under normoxic conditions; and
exposing the 3D cardiac organoid to hypoxic conditions for about 1 day to 20 days;
thereby generating the 3D myocardial infarct organoid.

10. A method of making a 3D myocardial ischemia-reperfused organoid, the method comprising:
culturing cardiomyocytes with non-myocytes for about 1 day to 20 days to form a 3D cardiac organoid under normoxic conditions;
exposing the 3D cardiac organoid under hypoxic conditions for about 1 day to 20 days to form a 3D myocardial infarct organoid, and
exposing the 3D myocardial infarct organoid to normoxic conditions for and/or exposing the 3D myocardial infarct organoid for fresh culture media about 5 seconds to 20 days.
thereby generating the 3D myocardial ischemia-reperfused organoid.

11. The method of claim 9, wherein the cardiomyocytes are cultured with the non-myocytes at a ratio of about 95:5 to about 5:95 of cardiomyocytes to non-my ocytes.

12. (canceled)

13. The method of claim 9, wherein the non-myocytes comprise fibroblasts (FBs), endothelial cells (ECs), mesenchymal stem cells (MSCs), or any combination thereof.

14. The method of claim 13, wherein the non-myocytes comprise FBs in amount of about 50% to 60% based on the total number of non-myocytes, ECs in an amount of about 25% to about 35% based on the total number of non-myocytes, and MSCs in an amount of about 10% to about 20% based on the total number of non-myocytes.

15. The method of claim 13, wherein the ECs comprise human umbilical vein endothelial cells (HUVECs) and/or the MSCs comprise human adipose derived stem cells (hADSCs).

16. The method of claim 9, wherein the cardiomyocytes and the non-myocytes are cultured at a total concentration of cardiomyocytes and non-myocytes of about 1×10⁵ cells/mL to about 1×10⁷ cells/mL.

17. The method of claim 9, wherein the cardiomyocytes and/or non-myocytes are from a human.

18. A 3D myocardial infarct organoid produced by the method of claim 9.

19. A 3D myocardial ischemia-reperfused organoid produced by the method claim 10.

20-24. (canceled)

25. A method for screening a compound for improving cardiac function, the method comprising:
contacting the 3D myocardial infarct organoid of claim 1 with the compound;
measuring in the 3D myocardial infarct organoid or the 3D myocardial ischemia-reperfused organoid the size of an apoptotic interior region, a ratio of a terminal deoxynucleotidyl transferase dUTP nick end labeling (TUNEL)-positive area to a 4′,6-diamidino-2-phenylindole (DAPI)-positive area in the apoptotic region, a contraction amplitude, a beat rate, a calcium transient amplitude, and/or an elastic modulus; and
determining that the compound improves cardiac function when
(a) the interior apoptotic region is reduced by at least about 30% when compared a control;
(b) the ratio of TUNEL-positive area to DAPI-positive area is reduced by at least about 30% when compared to a control;
(c) the contraction amplitude is increased by at least about 30% when compared to a control;
(d) the calcium transient amplitude is increased by at least about 30% when compared to a control; and/or
(e) the elastic modulus is decreased by at least about 30% when compared to a control;
wherein the control in (a)-(e) is the 3D myocardial infarct organoid of claim 1 that is not contacted with the compound.

25-26. (canceled)

27. A method for screening a compound for diminishing cardiac function, the method comprising:
contacting the 3D myocardial infarct organoid of claim 1 with the compound;
measuring in the 3D myocardial infarct organoid or the 3D myocardial ischemia-reperfused organoid the size of an apoptotic interior region, a ratio of a terminal deoxynucleotidyl transferase dUTP nick end labeling (TUNEL)-positive area to a 4′,6-diamidino-2-phenylindole (DAPI)-positive area in the apoptotic region, a contraction amplitude, a beat rate, a calcium transient amplitude, and/or an elastic modulus; and
determining that the compound diminishes cardiac function when
(a) the interior apoptotic region is increased by at least about 30% when compared a control;
(b) the ratio of TUNEL-positive area to DAPI-positive area is increased by at least 30% when compared to a control;
(c) the contraction amplitude is decreased by at least about 30% when compared to a control;
(d) the calcium transient amplitude is decreased by at least 30% when compared to a control; and/or
(e) the elastic modulus is increased by at least about 30% when compared to a control;
wherein the control in (a)-(e) is the 3D myocardial infarct organoid of claim 1 that is not contacted with the compound.
